(19) United States
(12) Patent Application Publication
(10) Pub. No.: US 2025/0268587 A1
(43) Pub. Date: Aug. 28, 2025

(54) LOCKING MECHANISM FOR MEDICAL DEVICE DELIVERY

(71) Applicant: EDWARDS LIFESCIENCES CORPORATION, Irvine, CA (US)

(72) Inventors: Zachary Charles VanEvery, Irvine, CA (US); Jonathan Andrew Lam, Anaheim Hills, CA (US); Ajay Kumar Dass, Costa Mesa, CA (US)

(21) Appl. No.: 18/587,610

(22) Filed: Feb. 26, 2024

Related U.S. Application Data
(63) Continuation of application No. PCT/US2022/040604, filed on Aug. 17, 2022.
(60) Provisional application No. 63/238,044, filed on Aug. 27, 2021.

(51) Int. Cl. A61B 17/00; A61M 25/00

(52) U.S. Cl. CPC A61B 17/00234; A61B 2017/00243; A61B 2017/00367; A61M 2025/0006

(57) ABSTRACT

A sheath assembly can include a hub that includes a housing with a delivery lumen extending through it. The delivery lumen can receive a portion of a medical device delivery assembly. A seal can be disposed within the housing. The seal can include a longitudinal lumen which forms a part of the delivery lumen of the housing. The seal can form a hemostatic seal around a corresponding portion of the medical device delivery assembly extending through the longitudinal lumen. An integrated rotational and translational lock can have a portion disposed within the housing proximally of the seal, the rotational and translational lock being able to, in a locked state, engage with a corresponding portion of the medical device delivery assembly extending through it to prevent rotational and translational movement of the medical device delivery assembly relative to the sheath assembly.

### CROSS REFERENCE TO RELATED APPLICATIONS

This application is a continuation of International Patent Application No. PCT/US2022/040604, filed Aug. 17, 2022, entitled “LOCKING MECHANISM FOR MEDICAL DEVICE DELIVERY,” which claims the benefit of U.S. Provisional Patent Application No. 63/238,044, filed Aug. 27, 2021, entitled “LOCKING MECHANISM FOR MEDICAL DEVICE DELIVERY,” the disclosures of each of which are hereby expressly incorporated by reference herein in their entireties for all purposes.

### BACKGROUND

### Field

The present disclosure generally relates to the field of minimally invasive delivery of medical devices.

### Description of Related Art

Minimally invasive procedures can involve transcatheter delivery of medical devices to a target site within the body, including within the heart. The minimally invasive procedures can be utilized to treat any number of conditions. Instability during the transcatheter delivery process can negatively impact outcomes of the procedures.

### SUMMARY

Described herein are methods and devices relating to medical delivery systems which can comprise a sheath assembly, a medical device loader and/or a locking collar, where each of the sheath assembly, medical device loader and locking collar comprises a rotational and translational lock configured to reduce or prevent rotational and translational movement of a medical device delivery assembly extending therethrough. The sheath assembly or medical device loader can comprise a hub that includes an integrated rotational and translational lock at least partially disposed within a housing of the hub and positioned proximally of a seal disposed within the housing. The rotational and translational lock of the locking collar can be at least partially disposed within a housing of the locking collar.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

For purposes of summarizing the disclosure, certain aspects, advantages and novel features have been described herein. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements. However, it should be understood that the use of similar reference numbers in connection with multiple drawings does not necessarily imply similarity between respective examples associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some examples or configurations.

FIG. 1 is a diagram showing features of a portion of the human vasculature, including the heart.

FIG. 2 is a longitudinal cross-sectional view of an example of a sheath assembly comprising a hub that includes an integrated rotational and translational lock comprising a spring-loaded contact member configured to contact a medical device delivery assembly extending through the sheath assembly.

FIG. 3 is a longitudinal cross-sectional view of an example of a sheath assembly comprising a hub that includes an integrated rotational and translational lock comprising a threaded rod member configured to contact a medical device delivery assembly extending through the sheath assembly.

FIGS. 4A, 4B, 4C and 4D show various views of an example of a sheath assembly comprising a hub that includes an integrated rotational and translational lock comprising a twistable tubular member configured to contact a medical device delivery assembly extending through the sheath assembly.

FIGS. 5A and 5B are longitudinal cross-sectional view of an example of a sheath assembly comprising a hub that includes an integrated rotational and translational lock comprising a rotatable proximal portion configured to contact a medical device delivery assembly extending through the sheath assembly.

FIGS. 6A and 6B are perspective views of an example of a sheath assembly comprising a hub that includes an integrated rotational and translational lock comprising a strap configured to contact a medical device delivery assembly extending through the sheath assembly.

FIGS. 7A and 7B are longitudinal cross-sectional views of an example of a sheath assembly comprising a hub that includes an integrated rotational and translational lock comprising an inflatable member configured to contact a medical device delivery assembly extending through the sheath assembly.

FIG. 8 is a longitudinal cross-sectional view of an example of a medical device loader comprising a hub that includes an integrated rotational and translational lock comprising a spring-loaded contact member configured to contact a medical device delivery catheter extending through the medical device loader.

FIG. 9 is a longitudinal cross-sectional view of an example of a medical delivery system comprising a sheath assembly and a locking collar comprising a rotational and translational lock.

FIG. 10 is a longitudinal cross-sectional view of an example of a locking collar comprising a rotational and translational lock.

FIG. 11A is a perspective view of a medical delivery system comprising a sheath assembly and the locking collar of FIG. 10. FIG. 11B is a longitudinal cross-sectional view of the engagement between the locking collar and the sheath assembly.

FIG. 12 is a flow diagram of an example of a process for deploying a medical device using a sheath assembly comprising an integrated rotational and translational lock.

FIG. 13 is a flow diagram of an example of a process for deploying a medical device using a medical delivery system comprising a locking collar comprising a rotational and translational lock.

### DETAILED DESCRIPTION

The headings provided herein are for convenience only and do not necessarily affect the scope or meaning of the claimed invention.

The present disclosure provides systems, devices, and methods relating to medical delivery systems which can comprise a sheath assembly, a medical device loader and/or a locking collar, where each of the sheath assembly, medical device loader and locking collar comprises a rotational and translational lock. A hub of the sheath assembly or medical device loader can comprise an integrated rotational and translational lock. The rotational and translational lock of the locking collar can be at least partially disposed within a housing of the locking collar. The locking collar can be used in combination with a sheath assembly or a medical device loader, for example engaging with a proximal portion, including a proximal end, of a sheath assembly hub or a medical device loader hub.

Although certain preferred examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to the preferred examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

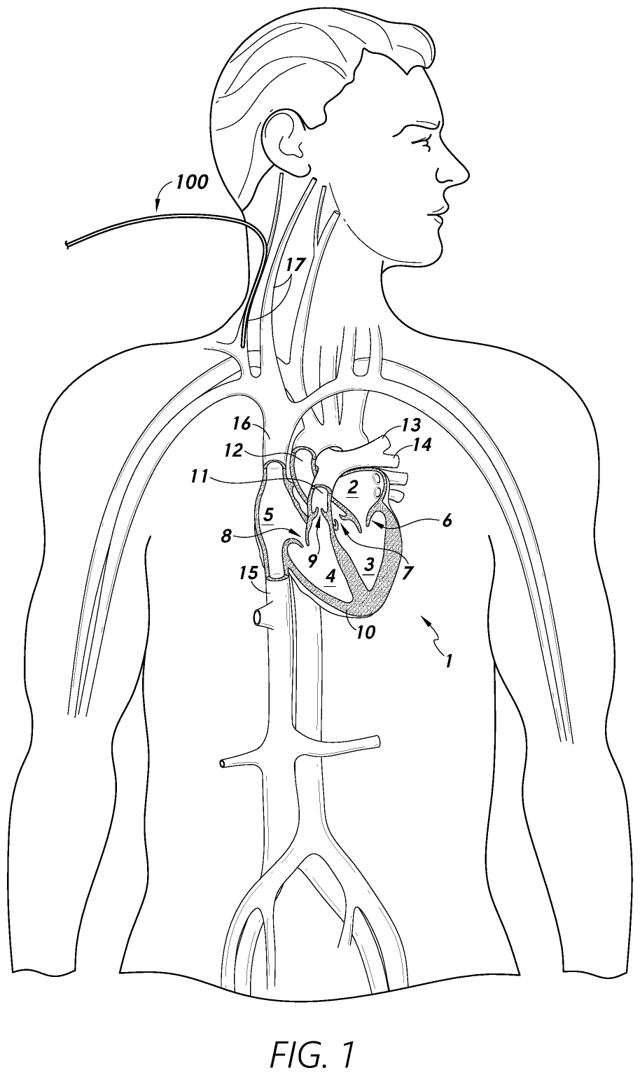

FIG. 1 shows certain anatomical features of human vasculature, including various features of a human heart 1. The heart 1 includes four chambers, namely the left atrium 2, the left ventricle 3, the right ventricle 4, and the right atrium 5. A wall of muscle, referred to as the septum 10, separates the left atrium 2 and right atrium 5, and the left ventricle 3 and right ventricle 4. Blood flow through the heart 1 is at least partially controlled by four valves, the mitral valve 6, aortic valve 7, tricuspid valve 8, and pulmonary valve 9. The mitral valve 6 separates the left atrium 2 and the left ventricle 3 and controls blood flow therebetween. The aortic valve 7 separates and controls blood flow between the left ventricle 3 and the aorta 12. The tricuspid valve 8 separates the right atrium 5 and the right ventricle 4 and controls blood flow therebetween. The pulmonary valve 9 separates the right ventricle 4 and the pulmonary trunk or artery 11, controlling blood flow therebetween.

In a healthy heart, the heart valves can properly open and close in response to a pressure gradient present during various stages of the cardiac cycle (e.g., relaxation and contraction) to at least partially control the flow of blood to a respective region of the heart and/or to blood vessels. Deoxygenated blood arriving from the rest of the body generally flows into the right side of the heart for transport to the lungs, and oxygenated blood from the lungs generally flows into the left side of the heart for transport to the rest of the body. During ventricular diastole, deoxygenated blood arrive in the right atrium 5 from the inferior vena cava 15 and superior vena cava 16 to flow into the right ventricle 4, and oxygenated blood arrive in the left atrium 2 from the pulmonary veins to flow into the left ventricle 3. During ventricular systole, deoxygenated blood from the right ventricle 4 can flow into the pulmonary trunk 11 for transport to the lungs (e.g., via the left 14 and right 13 pulmonary arteries), and oxygenated blood can flow from the left ventricle 3 to the aorta 12 for transport to the rest of the body.

A number of conditions can result in the need for surgical intervention, such as to deliver medical devices to various sites in and/or adjacent to the heart. Conditions such as mitral valve regurgitation and/or stenosis may result in difficulty in pumping blood from the left atrium to the left ventricle, contributing to elevated pressure in the left atrium. Enlargement of the heart can result, for example producing symptoms such as shortness of breath during exertion, fatigue, chest pain, fainting, abnormal heartbeat, and swelling of the legs and feet. Minimally invasive procedures involving transcatheter delivery of medical devices and/or therapy to a target site can be performed to ameliorate the conditions. In some cases, the minimally invasive procedures can involve a trans-jugular approach, requiring insertion of medical devices through the jugular vein 17. For example, as shown in FIG. 1, a medical delivery system 100 can be inserted into the vascular system through the jugular vein 17.

Instability of a medical delivery system during delivery of the medical device can adversely impact outcome of the procedure. The medical device can undesirably rotate about its longitudinal axis and/or translate proximally and distally. Typical medical delivery systems may require an operator to manually use one hand to maintain a desired position and/or orientation of the delivery system. Requiring the operator to hold onto the delivery system can limit tasks the operator can perform and/or slow down the procedure. In some cases, availability of footprint around the operating table may reduce the ability of an operator to control the stability of the delivery system and/or require the operator to stand at locations which expose the operator to radiation when fluoroscopy is used during the procedure. For example, footprint around the head of the operating table may be limited, restricting movement of the operator and/or locations at which the operator can stand when performing procedures involving access on, near, and/or around the neck, including for example procedures using the trans-jugular approach.

The disclosure herein provides one or more devices and methods related to medical delivery systems which can comprise a sheath assembly, a medical device loader and/or a locking collar, where each of the sheath assembly, medical device loader and locking collar can comprise a rotational and translational lock. The sheath assembly, medical device loader and/or locking collar can each comprise the rotational and translational lock integrated as a part thereof. In some instances, the sheath assembly can comprise a hub comprising an integrated rotational and translational lock configured to prevent rotation and translation of a medical device delivery assembly received by the sheath assembly. The integrated rotational and translational lock can be at least partially disposed within a housing of the hub and positioned proximally of a hemostatic seal disposed within the housing. In some instances, the integrated rotational and translational lock can be entirely or substantially entirely disposed within a housing of the hub. In a locked state, the integrated rotational and translational lock can be configured to engage a portion of the medical device delivery assembly extending through the lock such that the medical device delivery assembly is prevented from both rotating about a longitudinal axis of the medical device delivery assembly and translating proximally and distally relative to the sheath assembly. In some instances, frictional contact between the integrated rotational and translational lock and the medical device delivery assembly can be configured to prevent the rotational and translational movement of the delivery assembly relative to the sheath assembly. In an unlocked state, the integrated rotational and translational lock can be configured to allow the medical device delivery assembly to move freely relative to the sheath assembly.

In some instances, the medical device delivery assembly can comprise a medical device loader configured to receive a medical device delivery catheter. For example, at least a portion of medical device delivery catheter can be received by a delivery lumen of a medical device loader. The medical device loader can be received by a delivery lumen of a sheath assembly. The integrated rotational and translational lock of the sheath assembly can be configured to engage with a corresponding portion of the medical device loader. In some instances, the medical device delivery assembly comprises the medical device delivery catheter. In some instances, a medical device loader may not be used. For example, the medical device delivery catheter can be received directly by a sheath assembly. The integrated rotational and translational lock of the sheath assembly can be configured to engage with a corresponding portion of the medical device delivery catheter.

In some instances, the medical device loader can comprise a hub that includes an integrated rotational and translational lock. For example, the integrated rotational and translational lock can be at least partially disposed within a housing of a hub of the medical device loader and positioned proximally of a hemostatic seal disposed within the housing. In a locked state, the integrated rotational and translational lock can be configured to engage a portion of the medical device delivery catheter extending through the lock such that the medical device delivery catheter is prevented from both rotating about a longitudinal axis of the medical device delivery catheter and translating proximally and distally relative to the medical device loader. In an unlocked state, the integrated rotational and translational lock can be configured to allow the medical device delivery catheter to move freely relative to the medical device loader.

In some instances, the medical delivery system can comprise a sheath assembly configured to receive a medical device loader. The medical device loader can be configured to receive a medical device delivery catheter. In some instances, the medical device can be directly received within the medical device delivery catheter. A hub of the sheath assembly can comprise an integrated rotational and translational lock configured to engage with a portion of the medical device loader. The medical device loader can be prevented from rotating and translating relative to the sheath assembly. A hub of the medical device loader can comprise an integrated rotational and translational lock configured to engage with a portion of the medical device delivery catheter. The medical device delivery catheter can be prevented from rotating and translating relative to the medical device loader.

In some instances, a locking collar comprising a rotational and translational lock can be used in combination with a sheath assembly to prevent or reduce rotational and translational movement of a medical device delivery assembly received by the sheath assembly. For example, a medical delivery system can comprise a sheath assembly and a locking collar that comprises a rotational and translational lock. The locking collar can engage with the sheath assembly, such as a hub of the sheath assembly, such that the rotational orientation and translational position of the locking collar is fixed relative to the sheath assembly. In the locked state, the rotational and translational lock of the locking collar can engage with the medical device delivery assembly to prevent rotational and translational movement of the medical device delivery assembly relative to the locking collar. In some instances, frictional contact between the rotational and translational lock and the medical device delivery assembly can be configured to prevent the rotational and translational movement of the delivery assembly. In an unlocked state, the rotational and translational lock can be configured to allow the medical device delivery assembly to move freely relative to the locking collar. In some instances, a locking collar can be used in combination with a medical device loader, for example engaging with a proximal portion of a hub of the medical device loader.

The sheath assembly, medical device loader and/or locking collar as described herein can improve the stability of medical device delivery and/or medical therapy delivery. The sheath assemblies, medical device loaders and/or locking collars can reduce required footprint around the operating table, not require an operator to manually stabilize the delivery, and/or allow the operator to be stationed relative to the operating table to thereby reduce radiation exposure when fluoroscopy is used. The sheath assemblies, medical device loaders and/or locking collars can advantageously facilitate performance of procedures using access on, near, and/or around the neck, including for example procedures using the trans-jugular approach. In some instances, the sheath assemblies, medical device loaders and/or locking collars can advantageously facilitate performance of procedures using the trans-subclavian approach.

In some instances, the medical device can be delivered into the heart for treatment of various heart conditions. Although the medical device is described primarily herein as be used to address and/or ameliorate various heart conditions, it will be understood that the medical device can be any number of other instruments suited for delivery using the removable sheath assembly. The medical device can be delivered to any number of bodily lumens, vessels, chambers and/or cavities. The medical device can comprise any number of medical implant devices, surgical instruments, and/or medical devices which are temporarily positioned within the body to deliver various therapies. Although the sheath assemblies, medical device loaders and/or locking collars are described primarily as being used to facilitate transcatheter access through a location on, near, and/or around the neck, the sheath assemblies, medical device loaders and/or locking collars can be used to facilitate transcatheter access through any number of other access sites.

It will be understood that one or more components of the medical delivery systems described herein can undergo various processes in preparation for their use in various medical procedures, including for example sterilization processes. The sheath assemblies, medical device loaders and/or locking collars can be sterilized sheath assemblies, sterilized medical device loaders and/or sterilized locking collars, respectively.

The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

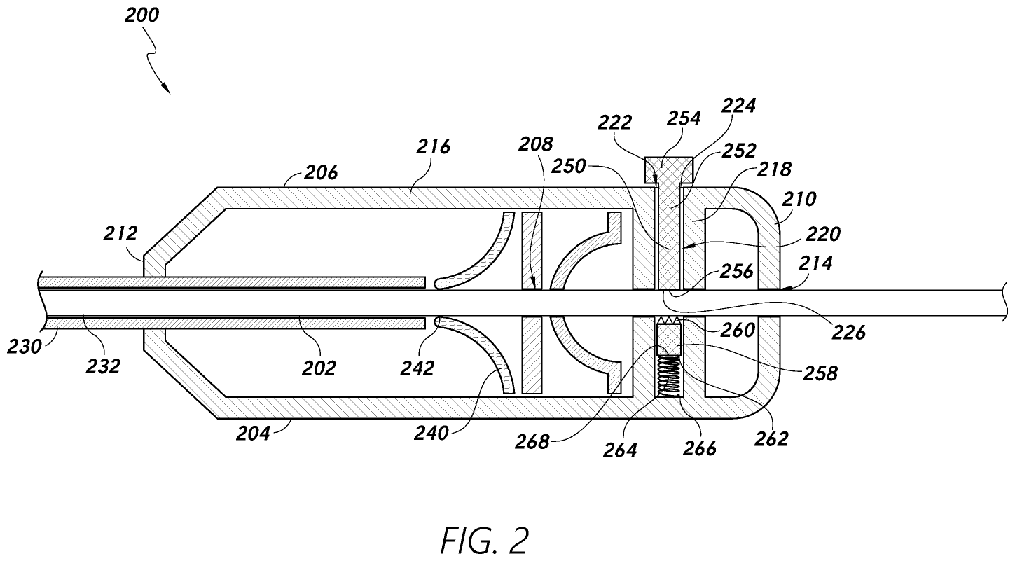

FIG. 2 is a longitudinal cross-sectional view of an example of a sheath assembly 200 comprising a hub 204 and a shaft 230, where the hub 204 can comprise an integrated rotational and translational lock 250. The sheath assembly 200 can comprise a delivery lumen 202 configured to receive at least a portion of a medical device delivery assembly. FIG. 2 shows a portion of the medical device delivery assembly being disposed through the delivery lumen 202 of the sheath assembly 200. In a locked state, the integrated rotational and translational lock 250 can be configured to engage with a portion of the medical device delivery assembly disposed in the delivery lumen 202 to prevent or reduce both change in rotational orientation and translational movement of the medical device delivery assembly relative to the sheath assembly 200. In some instances, the integrated rotational and translational lock 250 can prevent the medical device delivery assembly from both rotating about its longitudinal axis and translating distally and proximally relative to the sheath assembly 200.

The hub 204 can comprise a housing 206. The housing 206 can have a delivery lumen 208 extending therethrough. The delivery lumen 208 of the housing 206 can be parallel and/or coaxial with the longitudinal axis of the housing 206. The shaft 230 can comprise at least a portion that extends distally from a distal end 212 of the housing 206. For example, the shaft 230 can extend distally from a distal end 212 of the housing 206. The shaft 230 can comprise a delivery lumen 232 extending therethrough. The delivery lumen 232 of the shaft 230 can be aligned with the delivery lumen 208 of the housing 206. In some instances, the delivery lumen 232 of the shaft 230 can extend along a longitudinal axis of the shaft 230. In some instances, the delivery lumen 208 of the housing 206 can extend along a longitudinal axis of the housing 206. The delivery lumen 208 of the housing 206 and the delivery lumen 232 of the shaft 230 can form respective parts of the delivery lumen 202 of the sheath assembly 200. For example, corresponding portions of the medical device delivery assembly can extend through the delivery lumen 208 of the housing 206 and the delivery lumen 232 of the shaft 230. A portion of the medical device delivery assembly can extend through an opening 214 on a proximal end 210 of the housing 206 into the delivery lumen 208 of the housing 206, and into the delivery lumen 232 of the shaft 230 extending distally from the distal end 212 of the housing 206.

A seal 240 can be disposed within the housing 206. The seal 240 can be configured to form a hemostatic seal around a portion of the medical device delivery assembly extending through the seal 240. The seal 240 can include a longitudinal lumen 242 extending therethrough. In some instances, the longitudinal lumen 242 can comprise an opening extending through a longitudinal dimension of the seal 240, such as a dimension of the seal 240 parallel and/or coaxial with the longitudinal axis of the housing 206. The longitudinal lumen 242 can form a part of the delivery lumen 208 of the housing 206. A corresponding portion of the medical device delivery assembly can be configured to extend through the longitudinal lumen 242. In some instances, the seal 240 can be configured to form a hemostatic seal around a portion of the medical device delivery assembly extending through the longitudinal lumen 242.

The integrated rotational and translational lock 250 can be proximal of the seal 240. For example, a portion of the lock 250 can be disposed within the housing 206 proximally of the seal 240. The lock 250 can comprise an elongate lever 252 having an orientation perpendicular or substantially perpendicular to the longitudinal axis of the housing 206. For example, the elongate lever 252 having an orientation perpendicular or substantially perpendicular to the delivery lumen 208 of the housing 206. The elongate lever 252 can comprise a first user engagement end portion 254 extending externally of the housing 206 and a second delivery assembly engagement end portion 256 oriented toward the delivery lumen 208 of the housing 206. The lock 250 can comprise a spring-loaded contact member 258 comprising an engagement end 260 at an opposing position about the delivery lumen 208 relative to a position of the second delivery assembly engagement end portion 256 of the elongate lever 252. An opposing end 262 of the spring-loaded contact member 258 can be coupled to a spring 264. In some instances, a first end 266 of the spring 264 can be coupled to the housing 206 and a second opposing end 268 of the spring 264 can be coupled to the opposing end 262 of the spring-loaded contact member 258. While the spring 264 is in a relaxed state, the spring 264 can push the engagement end 260 of the spring-loaded contact member 258 against and maintain contact between the engagement end 260 and the medical device delivery assembly extending through the delivery lumen 208.

The elongate lever 252 can be in a depressed position or a released position. In the locked state, the elongate lever 252 can be in the depressed position such that the second delivery assembly engagement end portion 256 contacts a portion of the medical device delivery assembly extending through the delivery lumen 208 and push the medical device delivery assembly against the engagement end 260 of the spring-loaded contact member 258. Maintaining contact between the medical device delivery assembly and the second delivery assembly engagement end portion 256 of the elongate lever 252 and the second engagement end 260 of the spring-loaded contact member 258 can prevent or reduce rotational and translational movement of the medical device delivery assembly. In the unlocked state, the elongate lever 252 can be in a released position. While in the released position, the elongate lever 252 is not in contact with the medical device delivery assembly. The medical device delivery assembly can be allowed to move freely relative to the sheath assembly 200 in the unlocked state.

Referring to FIG. 2, the housing 206 can comprise a lateral housing portion 216. The lateral housing portion 216 can be parallel or substantially parallel to the longitudinal axis of the housing 206. The housing 206 can comprise a perpendicular housing portion 218 extending between the lateral housing portion 216 and the delivery lumen 208 of the housing 206. The perpendicular housing portion 218 can be perpendicular or substantially perpendicular to the longitudinal axis of the housing 206. The perpendicular housing portion 218 can define a perpendicular lumen 220. The perpendicular lumen 220 can extend between the lateral housing portion 216 and the delivery lumen 208 of the housing. A first end 224 of the perpendicular lumen 220 can be aligned with an opening 222 on the lateral housing portion 216 and a second end 226 of the perpendicular lumen 220 can open to the delivery lumen 208 of the housing 206.

The elongate lever 252 can be at least partially disposed within the perpendicular lumen 220. The first user engagement end portion 254 can extend externally of the housing 206 through the opening 222 on the lateral housing portion 216. The second delivery assembly engagement end portion 256 of the elongate lever 252 can be oriented toward the delivery lumen 208 of the housing 206 such that depressing the elongate lever 252 comprises advancing the second delivery assembly engagement end portion 256 toward the medical device delivery assembly and contacting a portion of the medical device delivery assembly disposed within the delivery lumen 208 of the housing 206 at the second end 226 of the perpendicular lumen 220. The elongate lever 252 can push the medical device delivery assembly onto the spring-loaded contact member 258 such that the medical device delivery assembly is securely sandwiched between the second delivery assembly engagement end portion 256 of the elongate lever 252 and the engagement end 260 of the spring-loaded contact member 258, preventing movement of the medical device delivery assembly. Releasing the elongate lever 252 can comprise displacing the second delivery assembly engagement end portion 256 away from the medical device delivery assembly to allow the medical device delivery assembly to move rotationally and/or translationally relative to the sheath assembly 200. In some instances, the elongate lever 252 can be coupled to a spring-loaded latch, such as a push latch, such that an operator can push on the first user engagement end portion 254 of the elongate lever 252 to advance the elongate lever 252 within the perpendicular lumen 220 to position the elongate lever 252 in the depressed state. For example, the operator pushing on the first user engagement end portion 254 can result in translating the elongate lever 252 toward the portion of the medical device delivery assembly disposed within the delivery lumen 208 of the housing 206 at the second end 226 of the perpendicular lumen 220. In some instances, pushing on the first user engagement end portion 254 can result in translating the elongate lever 252 toward the medical device delivery assembly such that the elongate lever 252 assumes the depressed state. The elongate lever 252 can be maintained in the depressed state, such as by the spring-loaded latch. Contact between the second delivery assembly engagement end portion 256 and the medical device delivery assembly can be maintained. The operator can subsequently push on the first user engagement end portion 254 again to position the elongate lever 252 in the released state. For example, pushing on the first user engagement end portion 254 again can result in translating the elongate lever 252 away from the medical device delivery assembly. The elongate lever 252 can be maintained in the released state, such as by the spring-loaded latch.

Alternatively, while in the unlocked state, the elongate lever 252 can be in the depressed state such that it is in contact with the spring-loaded contact member 258 to push the spring-loaded contact member 258 away from the portion of the medical device delivery assembly extending through the delivery lumen 208 of the housing 206. The second delivery assembly engagement end portion 256 of the elongate lever 252 can be in contact with the engagement end 260 of the spring-loaded contact member 258 such that the spring-loaded contact member 258 is spaced away from the medical device delivery assembly. The medical device delivery assembly can be allowed to freely rotate about its longitudinal axis and freely translate proximally and distally relative to the sheath assembly 200. In the locked state, the elongate lever 252 can be in the released state such that it is spaced from the spring-loaded contact member 258. The spring-loaded contact member 258 can be in contact with the portion of the medical device delivery assembly extending through the delivery lumen 208 of the housing 206. The second delivery assembly engagement end portion 256 of the elongate lever 252 can be spaced from the engagement end 260 of the spring-loaded contact member 258. For example, the engagement end 260 of the spring-loaded contact member 258 can be in contact with the medical device delivery assembly to push the assembly against one or more portions of the housing 206 defining the delivery lumen 208, thereby reducing or preventing its rotation and translation.

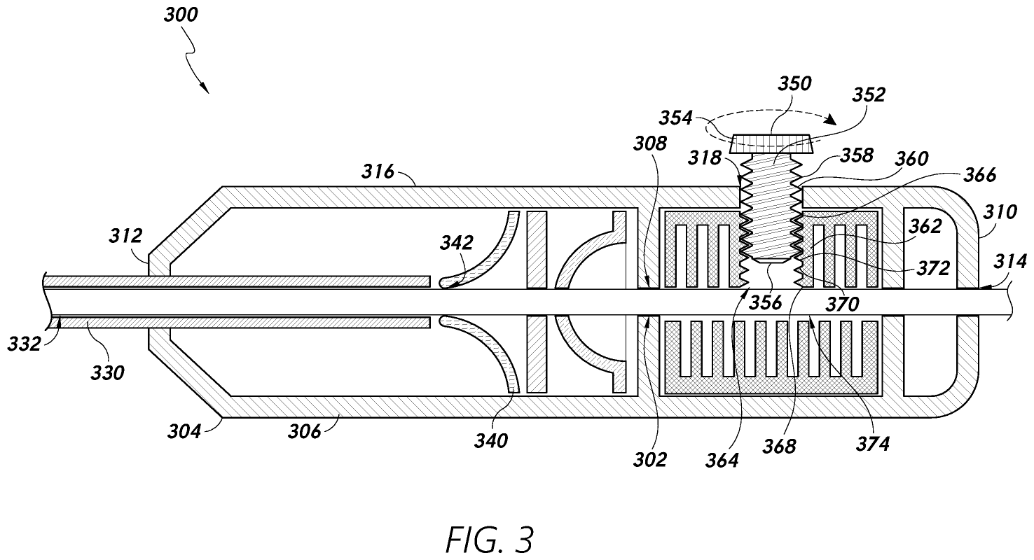

FIG. 3 is a longitudinal cross-sectional view of an example of a sheath assembly 300 with a hub 304 that has an integrated rotational and translational lock 350 comprising a threaded rod member 352. The sheath assembly 300 can comprise a delivery lumen 302 configured to receive at least a portion of a medical device delivery assembly. To achieve a locked state, the threaded rod member 352 can be configured to be rotated about its longitudinal in a first direction to advance the threaded rod member 352 toward a portion of the medical device delivery assembly until the threaded rod member 352 is in contact with the medical device delivery assembly. To achieve an unlocked state, the threaded rod member 352 can be rotated about its longitudinal axis in a second direction to withdraw the threaded rod member 352 away from the medical device delivery assembly such that the medical device delivery assembly can freely rotate about a longitudinal axis of the medical device delivery assembly and translate proximally and distally relative to the sheath assembly 300. FIG. 3 shows the integrated rotational and translational lock 350 in the unlocked state. A portion of the medical device delivery assembly is shown as being disposed through the delivery lumen 302 of the sheath assembly 300.

Referring to FIG. 3, the hub 304 can comprise a housing 306. The housing 306 can comprise a delivery lumen 308 extending through it. A seal 340 can be disposed within the housing 306. The seal 340 can include a longitudinal lumen 342 extending therethrough and forming a part of the delivery lumen 308 of the housing 306. In some instances, the longitudinal lumen 342 can comprise an opening extending through a longitudinal dimension of the seal 340, such as a dimension of the seal 340 parallel and/or coaxial with the longitudinal axis of the housing 306. A corresponding portion of the medical device delivery assembly can be configured to extend through the longitudinal lumen 342 such that the seal 340 can form a hemostatic seal around a portion of the medical device delivery assembly extending through the longitudinal lumen 342. The integrated rotational and translational lock 350 can be proximal of the seal 340. For example, a portion of the lock 350 can be disposed within the housing 306 proximally of the seal 340. A portion of the medical device delivery assembly can be positioned through an opening 314 on a proximal end 310 of the housing 306 and into the delivery lumen 308 of the housing 306. For example, the portion of the medical device delivery assembly disposed within the delivery lumen 308 of the housing 306 can comprise a portion disposed through the integrated rotational and translational lock 350 and another portion disposed through the longitudinal lumen 342 of the seal 340.

The integrated rotational and translational lock 350 can comprise a longitudinal lumen 374 extending therethrough and forming a portion of the delivery lumen 308 of the housing 306. A corresponding portion of the medical device delivery assembly can be disposed through the longitudinal lumen 374. For example, the threaded rod member 352 can engage with a portion of the medical device delivery assembly positioned through the longitudinal lumen 374. The threaded rod member 352 can have an orientation perpendicular or substantially perpendicular to the delivery lumen 308. For example, the delivery lumen 308 can be parallel or substantially parallel to a longitudinal axis of the housing 306. In some instances, the delivery lumen 308 can be coaxial with the longitudinal axis of the housing 306. The threaded rod member 352 can be perpendicular or substantially perpendicular to the longitudinal axis of the housing 306. The threaded rod member 352 can comprise a first user engagement end portion 354 configured to extend externally of the housing 306 and a second delivery assembly engagement end portion 356 oriented toward the delivery lumen 308 of the housing 306. Threads 358 on a lateral surface 360 of the threaded rod member 352 can be configured to mate with corresponding threads on a portion of the housing 306 and/or another portion of the lock 350. The first user engagement end portion 354 can be externally positioned through an opening 318 on a lateral housing portion 316. An operator, such as a surgeon, can manipulate the first user engagement end portion 354 to screw the threaded rod member 352 in the first direction, for example rotate the threaded rod member 352 about its longitudinal axis in the first direction, to cause contact between the second delivery assembly engagement end portion 356 and the medical device delivery assembly. Contact between the second delivery assembly engagement end portion 356 and the medical device delivery assembly can provide sufficient frictional force to reduce or prevent rotational and translational movement of the medical device delivery assembly. The operator can manipulate the first user engagement end portion 354 to screw the threaded rod member 352 in the second direction, for example rotate the threaded rod member 352 about its longitudinal axis in the second direction, to withdraw threaded rod member 352 and space apart the second delivery assembly engagement end portion 356 from the medical device delivery assembly, allowing rotation and translation of the medical device delivery assembly.

In some instances, the integrated rotational and translational lock 350 can comprise a perpendicular lock portion 362 which defines a perpendicular lumen 364 that is perpendicular or substantially perpendicular to the longitudinal axis of the housing 306. The perpendicular lock portion 362 can be perpendicular or substantially perpendicular to the longitudinal axis of the housing 306. The perpendicular lock portion 362 can have a first end 366 aligned with the opening 318 on the lateral housing portion 316 and a second end 368 opening to the delivery lumen 308 of the housing 306 such that the perpendicular lumen 364 can extend between the lateral housing portion 316 and the delivery lumen 308. An inner lumen surface 370 of the perpendicular lock portion 362 can comprise threads 372 along at least a portion thereof which are configured to mate with the threads 358 on the lateral surface 360 of the threaded rod member 352. The threaded rod member 352 can be partially disposed within the perpendicular lumen 364 such that the first user engagement end portion 354 can extend externally of the housing 306 through the opening 318 on the lateral portion 316 of the housing. The threaded rod member 352 can be rotated in the first direction about its longitudinal axis to advance the threaded rod member 352 within the perpendicular lumen 364 toward the medical device delivery assembly. The threaded rod member 352 can be rotated until contact is made between the medical device delivery assembly and the second delivery assembly engagement end portion 356 such that rotational and translational movement of the medical device delivery assembly is prevented. The threaded rod member 352 can push the medical device delivery assembly onto one or more portions of the integrated rotational and translational lock 350 defining the longitudinal lumen 374. The medical device delivery assembly can be securely sandwiched between the second delivery assembly engagement end portion 356 of the threaded rod member 352 and portions of the lock 350 defining the longitudinal lumen 374, thereby preventing rotational and translational movement of the medical device delivery assembly. The threaded rod member 352 can be rotated in the second direction about its longitudinal axis to displace the second delivery assembly engagement end portion 356 away from the medical device delivery assembly to allow the medical device delivery assembly to freely move rotationally and/or translationally relative to the sheath assembly 300.

In some instances, the threads 358 of the threaded rod member 352 and threads 372 on the inner lumen surface 370 defining the perpendicular lumen 364 can each comprise a pitch configured to facilitate manipulation of the integrated rotational and translational lock 350. In some instances, the threads 358 of the threaded rod member 352 and threads 372 on the inner lumen surface 370 defining the perpendicular lumen 364 can each comprise a pitch configured for a quarter turn in the first direction and the second direction to alternate between the locked state and the unlocked state, respectively.

Alternatively, in some instances, one or more portions of the housing 306 can define a perpendicular lumen configured to receive a portion of the threaded rod member 352. For example, the housing 306 can comprise a perpendicular housing portion extending between the lateral housing portion 316 and the delivery lumen 308 of the housing 306. A first end of the perpendicular housing portion can be aligned with the opening 318 on the lateral housing portion 316 and a second end of the perpendicular housing portion can open to the delivery lumen 308 of the housing 306. An inner lumen surface of the perpendicular housing portion defining perpendicular lumen can comprise threads to mate with threads 358 of the threaded rod member 352.

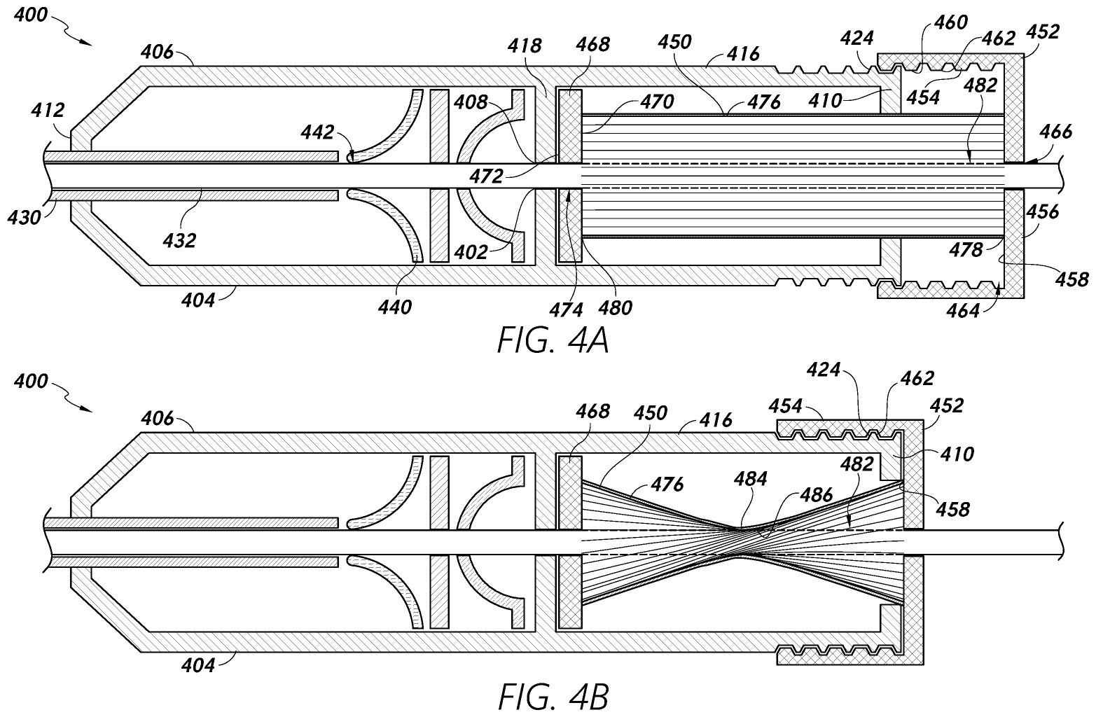

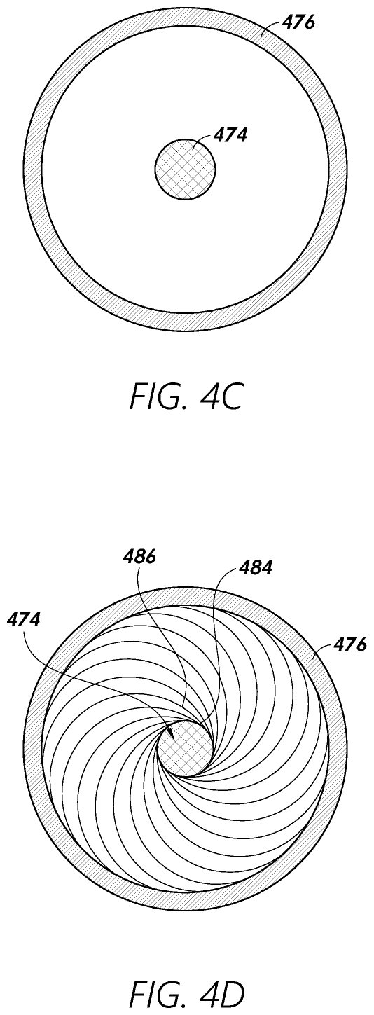

FIGS. 4A, 4B, 4C and 4D show various views of an example of a sheath assembly 400 comprising a hub 404 that includes an integrated rotational and translational lock 450 that has a tubular member 476 which can assume a twisted configuration to achieve a locked state and an untwisted configuration to achieve an unlocked state. FIGS. 4A and 4B are longitudinal cross-sectional views of the sheath assembly 400 with the integrated rotational and translational lock 450 in the unlocked state and the locked state, respectively. Each of the longitudinal cross-sectional views are taken along a plane extending along a longitudinal axis of the housing 406. FIGS. 4C and 4D show lateral cross-sectional views of the tubular member 476 in the unlocked and locked states, respectively. Each of the lateral cross-sectional views are taken along a plane perpendicular or substantially perpendicular to the longitudinal axis of the housing 406. The sheath assembly 400 can comprise a delivery lumen 402 configured to receive at least a portion of a medical device delivery assembly. A portion of a medical device delivery assembly is shown as being disposed through the delivery lumen 402. The tubular member 476 can comprise a longitudinal lumen 482 configured to form a portion of the delivery lumen 402 of the sheath assembly 400. In the locked state, an inner lumen surface 486 of the longitudinal lumen 482 in a twisted portion 484 of the tubular member 476 can be configured to contact a portion of the medical device delivery assembly extending therethrough to reduce or prevent rotational and translational movement of the medical device delivery assembly relative to the sheath assembly 400.

Referring to FIG. 4A, the hub 404 can comprise a housing 406. A seal 440 can be disposed within the housing 406. The seal 440 can include a longitudinal lumen 442 extending therethrough and forming a part of the delivery lumen 408 of the housing 406. In some instances, the longitudinal lumen 442 can comprise an opening extending through a longitudinal dimension of the seal 440, such as a dimension of the seal 440 parallel and/or coaxial with the longitudinal axis of the housing 406. A corresponding portion of the medical device delivery assembly can be configured to extend through the longitudinal lumen 442 such that the seal 440 can form a hemostatic seal around a portion of the medical device delivery assembly extending therethrough. The integrated rotational and translational lock 450 can be proximal of the seal 440. For example, a portion of the lock 450 can be disposed within the housing 406 proximally of the seal 440.

In some instances, the integrated rotational and translational lock 450 can comprise a proximal lock portion 452 that defines a distally oriented recess 464. The distally oriented recess 464 can be configured to receive a distal portion of the housing 406. The proximal lock portion 452 can comprise a lateral portion 454 and a proximal portion 456. In some instances, the lateral portion 454 can be oriented parallel or substantially parallel to the longitudinal axis of the housing 406. In some instances, the proximal portion 456 can be oriented perpendicularly or substantially perpendicularly to the longitudinal axis of the housing 406. The lateral portion 454 can extend distally from the proximal portion 456. A distally oriented surface 458 of the proximal portion 456, and a lateral surface 460 of the lateral portion 454 that is oriented toward the longitudinal axis of the housing 406, can at least partially define the recess 464. An opening 466 extending through the proximal portion 456 can be configured to allow extension therethrough of the medical device delivery assembly. The lateral surface 460 of the proximal lock portion 452 can comprise threads 462 configured to mate with corresponding threads 424 on the externally oriented surface of a lateral housing portion 416. The lock 450 can comprise a distal lock portion 468 disposed within the housing 406 and fixedly coupled to the housing 406. An opening 474 extending through the distal lock portion 468 can be configured to allow extension therethrough of the medical device delivery assembly. In some instances, the distal lock portion 468 can be coupled to a perpendicular housing portion 418 distal of the distal lock portion 468, the perpendicular housing portion being perpendicular or substantially perpendicular to the longitudinal axis of the housing 406. For example, a distal surface 472 of the distal lock portion 468 can be coupled to the perpendicular housing portion 418 such that the distal lock portion 468 has a rotational orientation and a translational position that is fixed and the same as that of the housing 406. Alternatively, or in combination, in some instances, the distal lock portion 468 can be coupled to a lateral housing portion 416 parallel or substantially parallel to the longitudinal axis of the housing 406.

The tubular member 476 can extend between and be coupled to the proximal and distal lock portions 452, 468. In some instances, a distal end 480 of the tubular member 476 can be coupled to a proximally oriented surface 470 of the distal lock portion 468 and a proximal end 478 of the tubular member 476 can be coupled to a distally oriented surface 458 of the proximal portion 456. A longitudinal lumen 482 extending through the tubular member 476 can form a portion of the delivery lumen 408 of the housing 406 such that a corresponding portion of the medical device delivery assembly can be positioned through the lock 450. The delivery lumen 408 can extend through the housing 406 and be parallel and/or coaxial with the longitudinal axis of the housing 406. For example, the medical device delivery assembly can extend through the opening 466 on the proximal portion 456 of the proximal lock portion 452, the longitudinal lumen 482 of the tubular member 476, and then through the opening 474 of the distal lock portion 468.

In some instances, the tubular member 476 can be coupled to a portion of the housing 406, such as without being coupled to a distal lock portion 468. For example, the integrated rotational and translational lock 450 may not have a distal lock portion 468 such that the tubular member 476 can be coupled directly to the perpendicular housing portion 418.

The proximal lock portion 452 can be rotated relative to the distal lock portion 468 and the housing 406 to achieve the locked and unlocked states. FIG. 4A shows the integrated rotational and translational lock 450 in the unlocked state. The tubular member 476 is in an untwisted state and the medical device delivery assembly is allowed to freely rotate and translate within the delivery lumen 408. The proximal lock portion 452 is shown as being partially screwed onto the housing 406 in the unlocked state.

FIG. 4B shows the integrated rotational and translational lock 450 in the locked state. The tubular member 476 can comprise a flexible and noncompliant material such that rotating the proximal lock portion 452 about the longitudinal axis of the housing 406 relative to the distal lock portion 468 is configured to cause the tubular member 476 to form a twisted portion 484. An inner lumen surface 486 defining the longitudinal lumen 482 of the tubular member 476 can be configured to contact the medical device delivery assembly extending through the twisted portion 484. In the locked state, the frictional force exerted upon the medical device delivery assembly by the twisted portion 484 can reduce or prevent rotational and translational movement of the medical device delivery assembly. Rotating the proximal lock portion 452 in the opposing direction about the longitudinal axis of the housing 406 can untwist the tubular member 476 to remove the twisted portion 484, such as shown in FIG. 4A. Untwisting the tubular member 476 can unlock the lock 450 to allow rotational and translational movement of the medical device delivery assembly. As the tubular member 476 is twisted to form the twisted portion 484, a length of the tubular member 476 is reduced such that the proximal portion 456 of the proximal lock portion 452 moves closer to the proximal end 410 of the housing 406. For example, to achieve the locked state the proximal lock portion 452 can be turned such that the proximal lock portion 452 is more screwed onto the housing 406. The distally oriented surface 458 of the proximal lock portion 452 can be closer to the proximal end 410 of the housing 406 in the locked state than while in the unlocked state. For example, in FIG. 4B, the proximal lock portion 452 is shown as being fully screwed onto the housing 406 in the locked state. In some instances, mating of the threads 462 on the proximal lock portion 452 and threads 424 the lateral housing portion 416 can facilitate maintaining the desired relative rotational displacement of the proximal lock portion 452 from the housing 406, such as to maintain the locked or unlocked state.

FIGS. 4C and 4D are lateral cross-sectional view of the tubular member 476 in the unlocked and locked states, respectively. The lateral cross section is taken along a plane perpendicular or substantially perpendicular to the longitudinal axis of the housing 406. In FIG. 4C, the tubular member 476 is not twisted. In FIG. 4D, the twisted portion 484 in the tubular member 476 is shown. As described herein, in the locked state, an inner lumen surface 486 defining the longitudinal lumen 482 of the tubular member 476 can be configured to contact the medical device delivery assembly extending through the twisted portion 484.

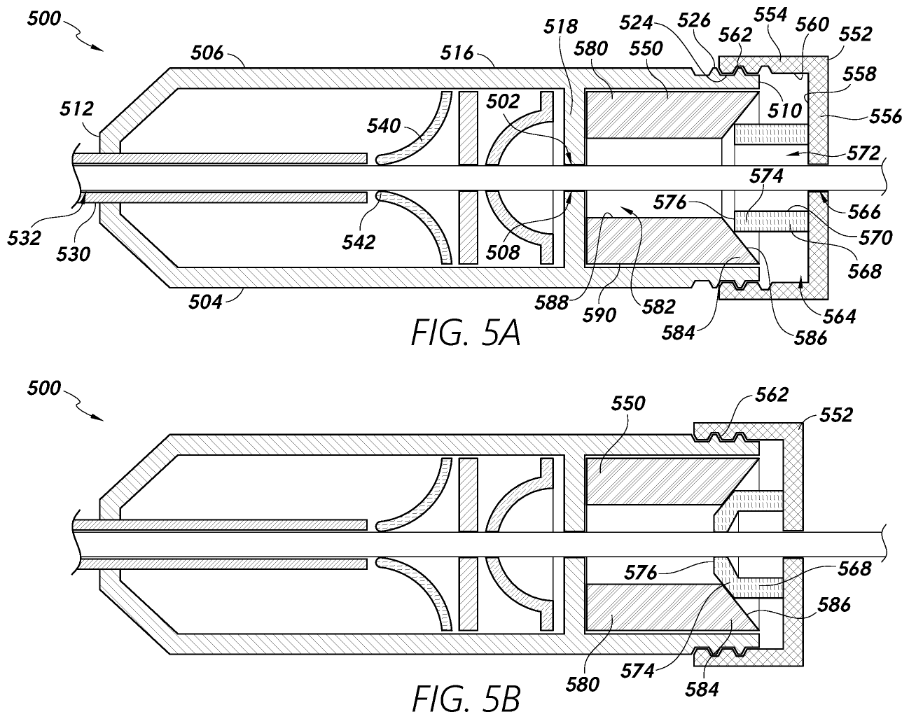

FIGS. 5A and 5B are longitudinal cross-sectional views of an example of a sheath assembly 500 comprising a hub 504 that includes an integrated rotational and translational lock 550 comprising a rotatable proximal portion configured to contact a medical device delivery assembly extending through the sheath assembly 500. The sheath assembly 500 can comprise a delivery lumen 502 configured to receive at least a portion of a medical device delivery assembly. FIG. 5A shows the integrated rotational and translational lock 550 in the unlocked state, where rotational and translational movement of the medical device delivery assembly is allowed. FIG. 5B shows the integrated rotational and translational lock 550 in the locked state, where rotational and translational movement of the medical device delivery assembly is reduced or prevented. A portion of the medical device delivery assembly is shown as being disposed through the delivery lumen 502.

Referring to FIGS. 5A and 5B, the hub 504 can comprise a housing 506. A seal 540 can be disposed within the housing 506. The seal 540 can include a longitudinal lumen 542 extending therethrough and forming a part of the delivery lumen 508 of the housing 506. In some instances, the longitudinal lumen 542 can comprise an opening extending through a longitudinal dimension of the seal 540, such as a dimension of the seal 540 parallel and/or coaxial with the longitudinal axis of the housing 506. A corresponding portion of the medical device delivery assembly can be configured to extend through the longitudinal lumen 542 such that the seal 540 can form a hemostatic seal around a portion of the medical device delivery assembly extending therethrough. The integrated rotational and translational lock 550 can be proximal of the seal 540. For example, a portion of the lock 550 can be disposed within the housing 506 proximally of the seal 540.

The integrated rotational and translational lock 550 can comprise a proximal lock portion 552 defining a distally oriented recess 564 configured to receive a distal portion of the housing 506. The proximal lock portion 552 can comprise a lateral portion 554 and a proximal portion 556. In some instances, the lateral portion 554 can be oriented parallel or substantially parallel to the longitudinal axis of the housing 506. In some instances, the proximal portion 556 can be oriented perpendicularly or substantially perpendicularly to the longitudinal axis of the housing 506. The lateral portion 554 can extend distally from the proximal portion 556. A distally oriented surface 558 of the proximal portion 556, and a lateral surface 560 of the lateral portion 554 that is oriented toward the longitudinal axis of the housing 506, can at least partially define the recess 564. An opening 566 extending through the proximal portion 556 can be configured to allow extension therethrough of the medical device delivery assembly. The lateral surface 560 of the proximal lock portion 552 can comprise threads 562 configured to mate with corresponding threads 524 on the externally oriented surface 526 of a lateral housing portion 516. The lateral housing portion 516 can be parallel or substantially parallel to a longitudinal axis of the housing 506. For example, the externally oriented surface 526 of the lateral housing portion 516 can be oriented away from, and the lateral surface 560 of the proximal lock portion 552 can be oriented toward, the delivery lumen 508 of the housing 506. The delivery lumen 508 of the housing 506 can extend through the housing 506 and be parallel and/or coaxial with the longitudinal axis of the housing 506. The proximal lock portion 552 can be rotated about the longitudinal axis of the housing 506 relative to the housing 506 to achieve the locked and unlocked states.

A distally extending member 568 can be coupled to the distally oriented surface 558 defining the recess 564. For example, the distally extending member 568 can extend distally from the distally oriented surface 558. The distally extending member 568 can comprise an inner lumen surface 570 which defines a first longitudinal lumen 572. The first longitudinal lumen 572 can form a portion of the delivery lumen 508 of the housing 506. For example, a proximal end of the first longitudinal lumen 572 can be aligned with the opening 566 of the proximal portion 556.

The integrated rotational and translational lock 550 can comprise a distal lock portion 580. The distal lock portion 580 can be disposed within the housing 506. The distal lock portion 580 can comprise a second longitudinal lumen 582 extending therethrough that forms another portion of the delivery lumen 508 of the housing 506. The second longitudinal lumen 582 can be aligned with the first longitudinal lumen 572. For example, the first and second longitudinal lumens 572, 582 can be coaxial with one another. In some instances, the first and second longitudinal lumens 572, 582 can be coaxial with the longitudinal axis of the housing 506. For example, the medical device delivery assembly can be advanced through the opening 566 on the proximal portion 556 of the proximal lock portion 552, through the first longitudinal lumen 572 of the distally extending member 568, and then through the second longitudinal lumen 582 of the distal lock portion 580.

The distal lock portion 580 can be fixedly coupled to the housing 506. The distal lock portion 580 can be coupled to the housing in any number of manners, including for example being coupled to the lateral housing portion 516 and/or perpendicular housing portion 518. Rotation of the proximal lock portion 552 in a first direction about the longitudinal axis of the housing 506 relative to the housing 506 can result in distally advancing the proximal lock portion 552 toward the housing 506. The proximal portion 556 of the proximal lock portion 552 can be moved toward the distal end 510 of the housing 506. For example, rotating the proximal lock portion 552 in the first direction can further screw the proximal lock portion 552 onto the housing 506. The distally extending member 568 of the proximal lock portion 552 can contact the distal lock portion 580 as the proximal lock portion 552 is advanced toward the distal lock portion 580 disposed in the housing 506. In some instances, a distal portion 574, including a distal end 576, of the distally extending member 568 can contact a proximal portion 584, including a proximal end 586, of the distal lock portion 580. Contact between the proximal lock portion 552 and the distal lock portion 580 can push the distal portion 574 of the proximal lock portion 552 inward toward the longitudinal axis of the housing 506 such that it can contact the medical device delivery assembly and achieve the locked state. For example, the distal end 576 of the proximal lock portion 552 can be bent toward the longitudinal axis of the housing 506 such that it contacts a portion of the medical device delivery assembly. Frictional contact between the distal portion 574 of the proximal lock portion 552 and the medical device delivery assembly can reduce or prevent its rotational and translational movement relative to the sheath assembly 500. Rotation of the proximal lock portion 552 about the second direction can proximally displace the proximal lock portion 552 so as to move the proximal portion 556 further away from the distal end 510 of the housing 506. For example, rotating the proximal lock portion 552 can unscrew the proximal lock portion 552. Bending of the distal portion 574 can be removed as the proximal portion 556 is moved away from the distal end of the housing, allowing the medical device delivery assembly to rotate and translate freely relative to the sheath assembly 500. In some instances, a diameter of the second longitudinal lumen 582 can be larger than that of the first longitudinal lumen 572 to facilitate bending inward of the distal portion 574.

In some instances, a proximal portion of the distal lock portion 580 can comprise a taper to facilitate bending inward of the distally extending member 568. The taper can facilitate bending of the distal portion 574 of the proximal lock portion 552 toward the longitudinal axis of the housing 506 when the distal portion 574 is in contact with the distal lock portion 580. In some instances, the proximal portion 584 of the distal lock portion 580 can comprise a taper that tapers inward toward the longitudinal axis of the housing 506. In some instances, an inner lumen surface 588 of the distal lock portion 580 defining the second longitudinal lumen 582 can have a shorter length than an opposing lateral surface portion 590 of the distal lock portion 580.

In some instances, at least a portion of the proximal lock portion 552 configured to be in contact with the medical device delivery assembly can comprise a material and/or texture configured to provide desired contact between the medical device delivery assembly and the proximal lock portion 552. In some instances, the distal portion 574, including the distal end 576, of the proximal lock portion 552 can comprise the material and/or texture. In some instances, at least a portion of the inner lumen surface 570 defining the first longitudinal lumen 572 can comprise the material and/or texture. In some instances, the texture can comprise one or more of ribs and/or dots. In some instances, the texture can comprise glass fibers.

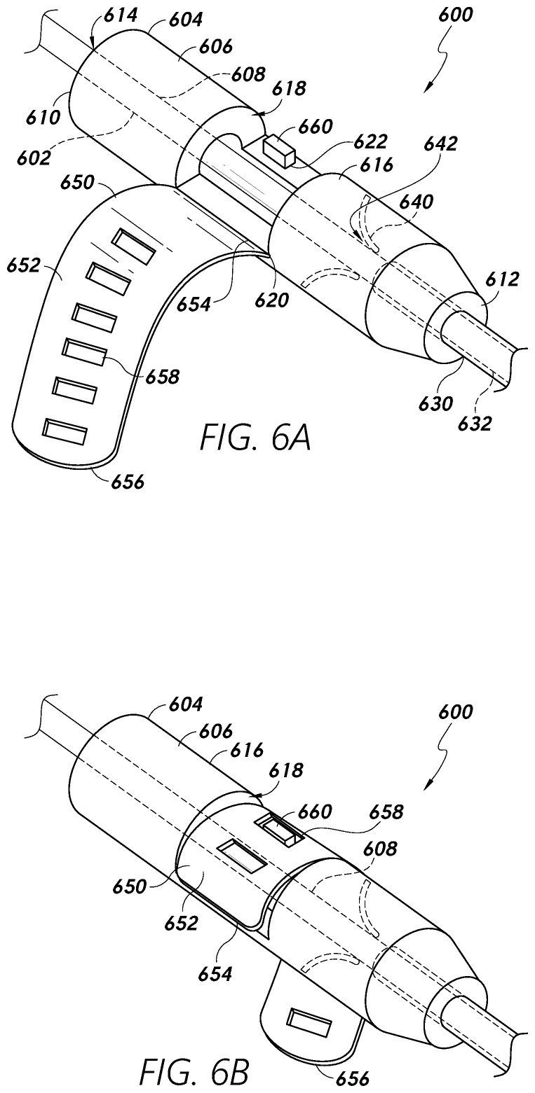

FIGS. 6A and 6B are perspective views of an example of a sheath assembly 600 that includes a hub 604 comprising an integrated rotational and translational lock 650 which has a strap 652. The sheath assembly 600 can comprise a delivery lumen 602 configured to receive at least a portion of a medical device delivery assembly. The strap 652 can be configured to contact a medical device delivery assembly extending through the sheath assembly 600 to reduce or prevent rotation and translation of the medical device delivery assembly. FIG. 6A shows the integrated rotational and translational lock 650 in the unlocked state, where rotational and translational movement of the medical device delivery assembly relative to the sheath assembly 600 is allowed. FIG. 6B shows the integrated rotational and translational lock 650 in the locked state, where rotational and translational movement of the medical device delivery assembly relative to the sheath assembly 600 is reduced or prevented. A portion of the medical device delivery assembly is shown as being disposed through the delivery lumen 602.

The hub 604 can comprise a housing 606. The housing 606 can comprise a delivery lumen 608 extending therethrough that is configured to receive a corresponding portion of the medical device delivery assembly. In some instances, the delivery lumen 608 can be parallel or substantially parallel to the longitudinal axis of the housing 606. In some instances, the delivery lumen 608 can be coaxial with the longitudinal axis of the housing 606. A proximal end 610 of the housing 606 can comprise an opening 614 thereon to allow extension therethrough of the medical device delivery assembly, such that the medical device delivery assembly can be advanced through the opening 614 into the delivery lumen 608. A seal 640 can be disposed within the housing 606 and be positioned distally of the integrated rotational and translational lock 650. The seal 640 can include a longitudinal lumen 642 extending therethrough and forming a part of the delivery lumen 608 of the housing 606. In some instances, the longitudinal lumen 642 can comprise an opening extending through a longitudinal dimension of the seal 640, such as a dimension of the seal 640 parallel and/or coaxial with the longitudinal axis of the housing 606. A corresponding portion of the medical device delivery assembly can be configured to extend through the longitudinal lumen 642 such that the seal 640 can form a hemostatic seal around a portion of the medical device delivery assembly extending therethrough.

Referring to FIGS. 6A and 6B, the housing 606 of the hub 604 can comprise a lateral housing portion 616 having an opening 618 extending therethrough. The lateral housing portion 616 can be parallel or substantially parallel to the longitudinal axis of the housing 606. The opening 618 can be in communication with the delivery lumen 608 of the housing 606 such that a portion of the medical device delivery assembly extending through the delivery lumen 608 can be exposed through the opening 618. The integrated rotational and translational lock 650 can comprise a strap 652 comprising a first end 654 configured to be associated with the housing 606 at a first position 620 on a first side of the opening 618 on the lateral housing portion 616 and a second free end 656. For example, the first end 654 can be coupled to the housing 606 at the first position 620. The strap 652 can comprise a plurality of fastening features 658 along at least a portion of a length of the strap 652. The fastening features 658 can be regularly spaced along the portion of the strap 652. The lock 650 can comprise a mating fastening feature 660 on the lateral housing portion 616 at a second position 622 on a second side of the opening 618. For example, the mating fastening feature 660 can be on a second side on the lateral housing portion 616 at an opposing position relative to that of the first end 654 of the strap 652. The first and second positions 620, 622 can be at opposing locations about the opening 618. In the locked state, the strap 652 can be at least partially wrapped around a lateral dimension of the housing 606 to position a portion of the strap 652 over the opening 618. The lateral dimension of the housing 606 can be perpendicular or substantially perpendicular to its longitudinal axis. A fastening feature 658 of the strap 652 can be coupled to the mating fastening feature 660 to provide the locked state. A portion of the strap 652 extending over the opening 618 on the lateral housing portion 616 can be configured to contact the exposed portion of medical device delivery assembly. Frictional contact between the strap 652 and the exposed portion of the medical device delivery assembly can be configured to prevent rotational and translational movement of the medical device delivery assembly relative to the sheath assembly 600. The unlocked state can be provided by loosening or unfastening the strap 652 such that the medical device delivery assembly can be allowed to rotate about its longitudinal axis and translate proximally and distally relative to the housing 606. For example, in the unlocked state, none of the fastening features 658 are coupled to the mating fastening feature 660.

In some instances, the mating fastening feature 660 can comprise a protrusion. A fastening feature 658 on the strap 652 can be an opening extending through a thickness of the strap 652. In some instances, the plurality of fastening features 658 can comprise a plurality of openings. For example, at least a portion of the protrusion can be configured to extend through an opening of the strap 652 in the locked state. The mating fastening feature 660 can comprise at least a portion configured to extend through a fastening feature 658 to provide the locked state. Alternatively, or in combination, any number of other fastening mechanisms can be applicable, including for example, a snap-on button type fastener, hook-and-loop fastener, and/or buckle fastener. In some instances, the strap 652 can comprise material and/or texture to provide desired frictional contact with the medical device delivery assembly. For example, a surface portion of the strap 652 configured to contact the medical device delivery assembly can comprise a material and/or texture to provide frictional contact with the medical device delivery assembly to prevent rotation and translation of the medical device delivery assembly.

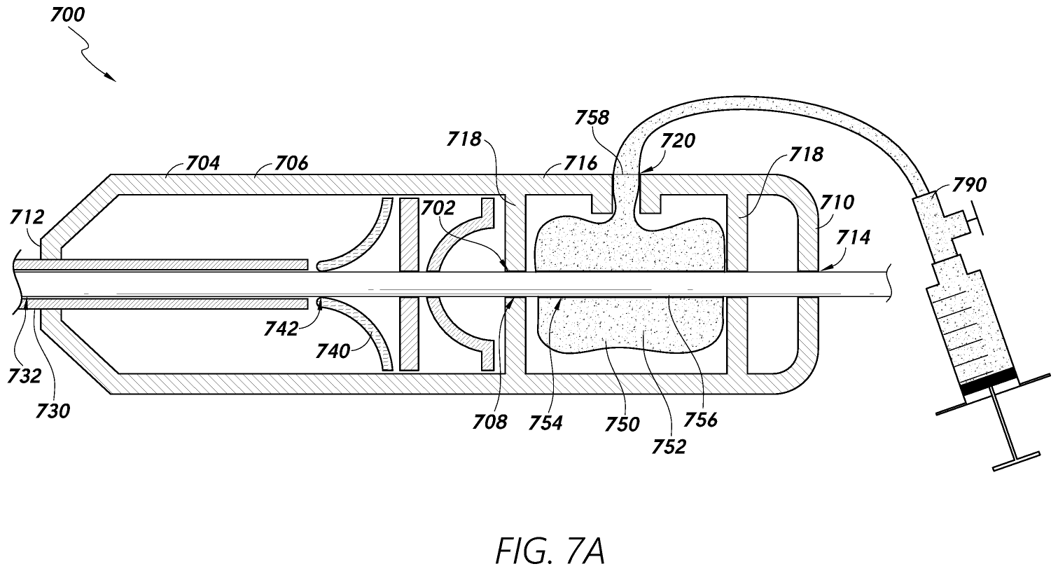

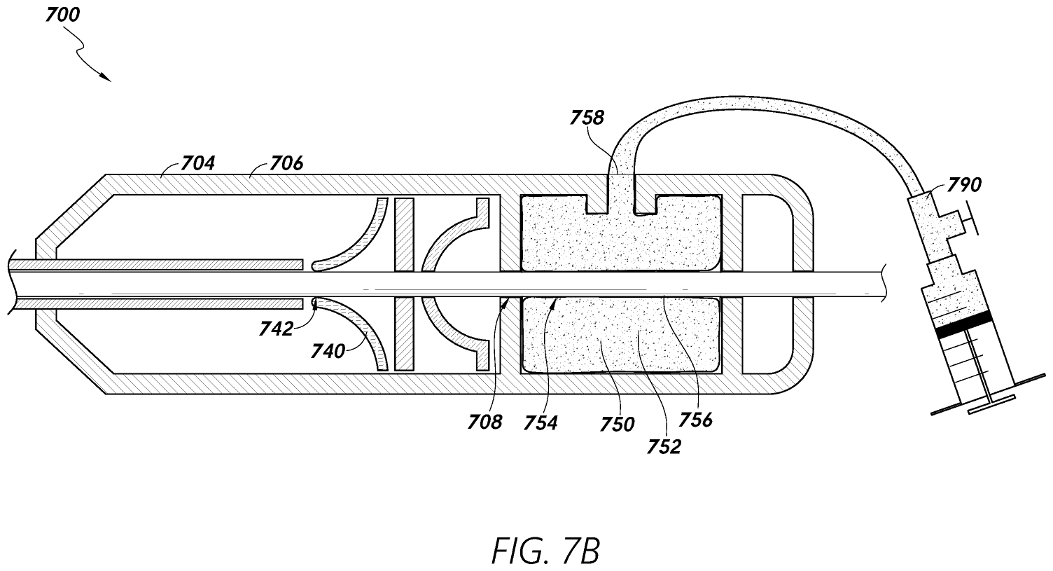

FIGS. 7A and 7B are longitudinal cross-sectional views of an example of a sheath assembly 700 that includes a hub 704 comprising an integrated rotational and translational lock 750 which has an inflatable member 752. The sheath assembly 700 can comprise a delivery lumen 702 configured to receive at least a portion of a medical device delivery assembly. The inflatable member 752 can be configured to be inflated so as to contact a medical device delivery assembly extending through the delivery lumen 702 of the sheath assembly 700. FIGS. 7A and 7B show a portion of the medical device delivery assembly disposed through the delivery lumen 702. Frictional contact between the inflatable member 752 and the medical device delivery assembly can reduce or prevent rotation and translation of the medical device delivery assembly. FIG. 7A shows the integrated rotational and translational lock 750 in the unlocked state, where rotational and translational movement of the medical device delivery assembly relative to the sheath assembly 700 is allowed. FIG. 7B shows the integrated rotational and translational lock 750 in the locked state, where rotational and translational movement of the medical device delivery assembly relative to the sheath assembly 700 is reduced or prevented.

The hub 704 can comprise a housing 706. The housing 706 can comprise a delivery lumen 708 extending therethrough that is configured to receive a corresponding portion of the medical device delivery assembly. In some instances, the delivery lumen 708 can be parallel or substantially parallel to the longitudinal axis of the housing 706. In some instances, the delivery lumen 708 can be coaxial with the longitudinal axis of the housing 706. A proximal end 710 of the housing 706 can comprise an opening 714 thereon to allow extension therethrough of the medical device delivery assembly, such that the medical device delivery assembly can be advanced through the opening 714 into the delivery lumen 708. A seal 740 can be disposed within the housing 706 and be positioned distally of the integrated rotational and translational lock 750. The seal 740 can include a longitudinal lumen 742 extending therethrough and forming a part of the delivery lumen 708 of the housing 706. In some instances, the longitudinal lumen 742 can comprise an opening extending through a longitudinal dimension of the seal 740, such as a dimension of the seal 740 parallel and/or coaxial with the longitudinal axis of the housing 706. A corresponding portion of the medical device delivery assembly can be configured to extend through the longitudinal lumen 742 such that the seal 740 can form a hemostatic seal around a portion of the medical device delivery assembly extending therethrough.

The inflatable member 752 of the integrated rotational and translational lock 750 can be disposed within the housing 706. The inflatable member 752 can define a longitudinal lumen 754 extending therethrough. The longitudinal lumen 754 can form a part of the delivery lumen 708 of the housing 706. For example, a corresponding portion of the medical device delivery assembly can be configured to be received within the longitudinal lumen 754. The inflatable member 752 can be inflated to provide the locked state. A diameter of the longitudinal lumen 754 can decrease as the inflatable member 752 is inflated. An inner lumen surface 756 of the inflatable member 752 can define the longitudinal lumen 754. Inflation of the inflatable member 752 can be continued until contact occurs between the inner lumen surface 756 of the inflatable member 752 and a portion of the medical delivery assembly extending through the longitudinal lumen 754. The inflatable member 752 can be inflated to an inflated state such that sufficient frictional contact is provided between the medical device delivery assembly and the inner lumen surface 756 to prevent or reduce rotational and translational movement of the medical device delivery assembly. In some instances, a degree of inflation of the inflatable member 752 can be adjusted to achieve the inflated state based on desired frictional contact between the inner lumen surface 756 and the medical device delivery assembly. In some instances, the degree of inflation of the inflatable member 752 can be adjusted based at least in part on a diameter of the medical device delivery assembly extending therethrough. The inflatable member 752 can be received or substantially received within the housing 706. In some instances, the inflatable member 752 can be received within a cavity defined at least in part by the lateral housing portion 716 and two perpendicular housing portions 718. Each of the perpendicular housing portions 718 can be perpendicular or substantially perpendicular to the longitudinal axis of the housing 706.

The inflatable member 752 can comprise an injection port 758 aligned with an opening 720 on the lateral housing portion 716. The injection port 758 can provide fluid communication with an inner volume of the inflatable member 752. The injection port 758 can be configured to mate with an injection device 790 that is used to inflate and/or deflate the inflatable member 752. A fluid can be provided into the inflatable member 752 to inflate the inflatable member 752 and/or withdrawn from the inflatable member 752 to deflate the inflatable member 752 using the injection device 790. The fluid can comprise a liquid or a gas. In some instances, the fluid can be a saline solution.

The sheath assemblies 300, 400, 500, 600, 700 described with reference to FIGS. 3, 4, 5, 6 and 7 can comprise one or more other features of the sheath assembly 200 describe with reference to FIG. 2. For example, the sheath assemblies 300, 400, 500, 600, 700 can each comprise a shaft 330, 430, 530, 630, 730 extending distally from a distal end 312, 412, 512, 612, 712 of the respective housing 306, 406, 506, 606, 706. The shafts 330, 430, 530, 630, 730 can each comprise a delivery lumen 332, 432, 532, 632, 732 extending therethrough, forming a portion of the respective delivery lumen 302, 402, 502, 602, 702 of the sheath assemblies 300, 400, 500, 600, 700 to received corresponding portions of the medical device delivery assembly. For example, the delivery lumens 332, 432, 532, 632, 732 of the shafts 330, 430, 530, 630, 730 can be aligned with, including being coaxial with, the respective delivery lumen 308, 408, 508, 608, 708 of the housing 306, 406, 506, 606, 706.

In some instances, the medical device delivery assembly can comprise a medical device loader configured to receive a medical device delivery catheter. For example, at least a portion of medical device delivery catheter can be received by a delivery lumen of a medical device loader. The medical device loader can be received by a delivery lumen of a sheath assembly described herein. The integrated rotational and translational lock of the sheath assembly can be configured to engage with a corresponding portion of the medical device loader. In some instances, the shaft of the sheath assembly may be collapsible. For example, the shaft of the sheath assembly can assume a collapsed state while no medical device delivery catheter and/or medical device loader is received therethrough. Force exerted upon the shaft by the medical device delivery catheter and/or medical device loader while passing the medical device delivery catheter and/or medical device loader through the shaft can cause respective portions of the shaft to expand. In some instances, the medical device loader can provide protection for the medical device delivery catheter and/or medical device during passage through the shaft. The medical device loader can be configured to provide additional structural protection for the medical delivery catheter extending therethrough.

In some instances, the medical device delivery assembly can comprise the medical device delivery catheter. For example, a medical device loader may not be used, such that the medical device delivery catheter is directly received by the sheath assembly. The integrated rotational and translational lock of the sheath assembly can be configured to engage with a corresponding portion of the medical device delivery catheter.

In some instances, the medical device loader can comprise a hub that includes an integrated rotational and translational lock. For example, the integrated rotational and translational lock can be at least partially disposed within a housing of a hub of the medical device loader and positioned proximally of a hemostatic seal disposed within the housing. In a locked state, the integrated rotational and translational lock can be configured to engage a portion of the medical device delivery catheter extending through the lock such that the medical device delivery catheter is prevented from both rotating about a longitudinal axis of the medical device delivery catheter and translating proximally and distally relative to the medical device loader. In an unlocked state, the integrated rotational and translational lock can be configured to allow the medical device delivery assembly to move freely relative to the medical device loader.

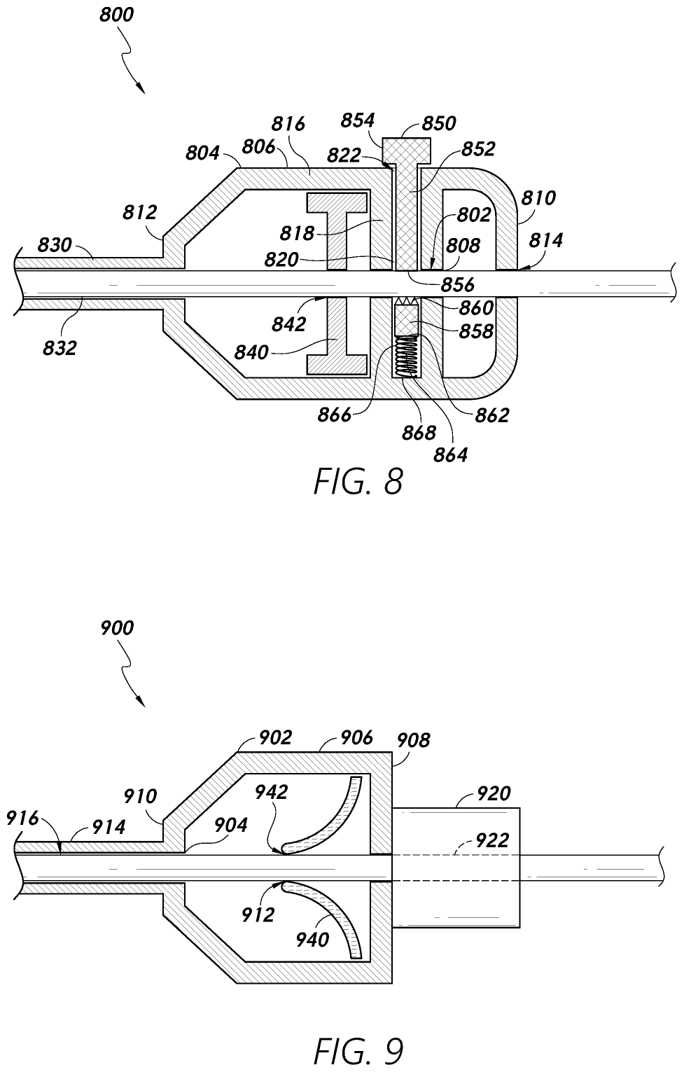

FIG. 8 is a longitudinal cross-sectional view of an example of a medical device loader 800 comprising a delivery lumen 802 configured to receive at least a portion of a medical device delivery catheter, where a hub 804 of the medical device loader 800 includes an integrated rotational and translational lock 850. FIG. 8 shows a portion of the medical device delivery catheter disposed through the delivery lumen 802. The integrated rotational and translational lock 850 can comprise a spring-loaded contact member 858 configured to contact a medical device delivery catheter extending through the medical device loader 800. In a locked state, the integrated rotational and translational lock 850 can be configured to engage with a portion of the medical device delivery catheter disposed in the delivery lumen 802 to prevent or reduce both change in rotational orientation and translational position of the medical device delivery catheter relative to the medical device loader 800. In an unlocked state, the medical device delivery catheter disposed in the delivery lumen 802 can be allowed to both rotate about a longitudinal axis of the medical device delivery catheter and translated proximal and distally relative to the medical device loader 800.

In some instances, at least a portion of the medical device loader 800 can be positioned through a sheath assembly to facilitate deployment of a medical device to a target site. For example, a medical delivery system can comprise both a sheath assembly and a medical device loader, such as the medical device loader 800. In some instances, both a rotational orientation and translational position of the medical device loader can be fixed relative to the sheath assembly and both a rotational orientation and translational position of the medical device delivery catheter can be fixed relative to the medical device loader. In some instances, the sheath assembly can comprise one or more configurations described herein.

Referring to FIG. 8, the hub 804 can comprise a housing 806. The housing 806 can have a delivery lumen 808 extending therethrough. In some instances, the delivery lumen 808 of the housing 806 can extend along a longitudinal axis of the housing 806. In some instances, the delivery lumen 808 can be parallel or substantially parallel to the longitudinal axis of the housing 806. A shaft 830 can extend distally from a distal end 812 of the housing 806. The shaft 830 can comprise a delivery lumen 832 extending therethrough. The delivery lumen 832 of the shaft 830 can be aligned with the delivery lumen 808 of the housing 806, for example being coaxial with the delivery lumen 808 of the housing 806. The delivery lumen 808 of the housing 806 and the delivery lumen 832 of the shaft 830 can form respective parts of the delivery lumen 802 of the medical device loader 800. For example, a portion of the medical device delivery catheter can extend through an opening 814 on a proximal end 810 of the housing 806 into the delivery lumen 808 of the housing 806, and into the delivery lumen 832 of the shaft 830 extending distally from the distal end 812 of the housing 806.

A seal 840 configured to form a hemostatic seal around a portion of the medical device delivery catheter can be disposed within the housing 806. The seal 840 can include a longitudinal lumen 842 extending therethrough. The longitudinal lumen 842 can form a part of the delivery lumen 808 of the housing 806. In some instances, the longitudinal lumen 842 can comprise an opening extending through a longitudinal dimension of the seal 840, such as a dimension of the seal 840 parallel and/or coaxial with the longitudinal axis of the housing 806. The seal 840 can be configured to form a hemostatic seal around a portion of the medical device delivery catheter extending through the longitudinal lumen 842.

The integrated rotational and translational lock 850 can be proximal of the seal 840. In some instances, a portion of the lock 850 can be disposed within the housing 806 proximally of the seal 840. The lock 850 can comprise an elongate lever 852 having an orientation perpendicular or substantially perpendicular to the longitudinal axis of the housing 806. For example, the elongate lever 852 can have an orientation perpendicular or substantially perpendicular to the delivery lumen 808 of the housing 806. The elongate lever 852 can comprise a first user engagement end portion 854 extending externally of the housing 806 and a second delivery catheter engagement end portion 856 oriented toward the delivery lumen 808 of the housing 806. The lock 850 can comprise the spring-loaded contact member 858 comprising an engagement end 860 at an opposing position about the delivery lumen 808 relative to a position of the second delivery catheter engagement end portion 856 of the elongate lever 852. An opposing end 862 of the spring-loaded contact member 858 can be coupled to a spring 864. In some instances, a first end 866 of the spring 864 can be coupled to the housing 806 and a second opposing end 868 of the spring 864 can be coupled to the opposing end 862 of the spring-loaded contact member 858. While the spring 864 is in a relaxed state, the spring 864 can push the engagement end 860 of the spring-loaded contact member 858 against the medical device delivery catheter extending through the delivery lumen 808.

In some instances, the elongate lever 852 can be in a depressed position such that the second delivery catheter engagement end portion 856 contacts a portion of the medical device delivery catheter extending through the delivery lumen 808 and pushes the medical device delivery catheter against the engagement end 860 of the spring-loaded contact member 858. Sandwiching the medical device delivery catheter between the second delivery catheter engagement end portion 856 and the second engagement end 860 can prevent or reduce rotational and translational movement of the medical device delivery catheter. In the unlocked state, the elongate lever 852 can be in a released position. While in the released position, the elongate lever 852 can be spaced from and not in contact with the medical device delivery catheter. The medical device delivery catheter can be allowed to rotate move freely relative to the medical device loader 800 in the unlocked state.

In some instances, the housing 806 can comprise a lateral housing portion 816. The lateral housing portion 816 can be parallel or substantially parallel to the longitudinal axis of the housing 806. The housing 806 can comprise a perpendicular housing portion 818 extending between the lateral housing portion 816 and the delivery lumen 808 of the housing 806. The perpendicular housing portion 818 can be perpendicular or substantially perpendicular to the longitudinal axis of the housing 806. The perpendicular housing portion 818 can define a perpendicular lumen 820 extending between the lateral housing portion 816 and the delivery lumen 808 of the housing 806. A first end of the perpendicular lumen 820 can be aligned with an opening 822 on the lateral housing portion 816 and a second end of the perpendicular lumen 820 can open to the delivery lumen 808 of the housing 806.

The elongate lever 852 can be at least partially disposed within the perpendicular lumen 820. The first user engagement end portion 854 can extend externally of the housing 806 through the opening 822 on the lateral housing portion 816. The second delivery catheter engagement end portion 856 of the elongate lever 852 can be oriented toward the delivery lumen 808 of the housing 806 such that depressing the elongate lever 852 comprises advancing the second delivery catheter engagement end portion 856 within the perpendicular lumen 820 toward the medical device delivery catheter and contacting the medical device delivery catheter. The elongate lever 852 can push the medical device delivery catheter onto the spring-loaded contact member 858 such that the medical device delivery catheter is securely sandwiched between the second delivery catheter engagement end portion 856 of the elongate lever 852 and the engagement end 860 of the spring-loaded contact member 858, preventing movement of the medical device delivery catheter. Releasing the elongate lever 852 can comprise withdrawing the second delivery catheter engagement end portion 856 away from the medical device delivery assembly, for example such that the second delivery catheter engagement end portion 856 is positioned within the perpendicular lumen 820.

Alternatively, the elongate lever 852 can be configured to contact the spring-loaded contact member 858 to provide the locked and unlocked states. For example, while in the unlocked state, the elongate lever 852 can be in the depressed state such that it is in contact with the spring-loaded contact member 858 to push the spring-loaded contact member 858 away from the portion of the medical device delivery catheter extending through the delivery lumen 808 of the housing 206. The second delivery catheter engagement end portion 856 of the elongate lever 852 can be in contact with the engagement end 860 of the spring-loaded contact member 858 such that the spring-loaded contact member 858 is spaced away from the medical device delivery catheter. While the spring-loaded contact member 858 is spaced away from the medical device delivery catheter, the medical device delivery catheter can be allowed to translate and rotate. In the locked state, the elongate lever 852 can be in the released state such that it is spaced from the spring-loaded contact member 858. The spring-loaded contact member 858 can be in contact with the portion of the medical device delivery catheter extending through the delivery lumen 808 of the housing 806 to push the catheter against one or more portions of the housing 806 defining the delivery lumen 808, thereby reducing or preventing its rotation and translation. For example, in the locked state, the second delivery catheter engagement end portion 856 of the elongate lever 852 can be spaced from the engagement end 860 of the spring-loaded contact member 858.

In some instances, the elongate lever 852 can be coupled to a spring-loaded latch, such as a push latch, such that an operator can push on the first user engagement end portion 854 of the elongate lever 852 to advance the elongate lever 852 into the perpendicular lumen 820 to position the elongate lever 852 in the depressed state. The operator can subsequently push on the first user engagement end portion 854 again to position the elongate lever 852 in the released state.

In some instances, a medical delivery system can comprise a sheath assembly configured to receive a medical device loader. One or more sheath assemblies described herein can be configured to receive a medical device loader, such as the medical device loader 800 described with reference to FIG. 8. For example, the sheath assembly 200, 300, 400, 500, 600, or 700 as described with reference to FIG. 2, 3, 4, 5, 6 or 7 can be configured to receive a medical device loader. The medical device loader can be configured to receive a medical device delivery catheter. The medical device can be directly received within the medical device delivery catheter. A hub of the sheath assembly can comprise an integrated rotational and translational lock as described herein and configured to engage with a portion of the medical device loader. The medical device loader can thereby be prevented from rotating and translating relative to the sheath assembly. A hub of the medical device loader can comprise an integrated rotational and translational lock configured to engage with a portion of the medical device delivery catheter to thereby prevent the medical device delivery catheter from rotating and translating relative to the medical device loader. The medical delivery catheter is thereby prevented from rotating and translating relative to the sheath assembly.

In some instances, a rotational and translational lock configured to fix a rotational orientation and translational position of a medical device delivery assembly received by a sheath assembly can be separate from the sheath assembly. A locking collar comprising a rotational and translational lock can be used in combination with a sheath assembly to prevent or reduce rotational and translational movement of the medical device delivery assembly. FIG. 9 shows an example of a medical delivery system 900 comprising a sheath assembly 902 and a locking collar 920 comprising a rotational and translational lock (not shown). The rotational and translational lock can be integrated as a part of the locking collar 920, for example at least partially housed within the locking collar 920. In some instances, the rotational and translational lock can be entirely housed within the locking collar 920. The sheath assembly 902 can comprise a delivery lumen 904 through which at least a portion of the medical device delivery assembly can be advanced. The sheath assembly 902 can comprise a hub 906 and a shaft 914 extending distally from a distal end 910 of the hub 906. The locking collar 920 can engage with the sheath assembly 902, including a proximal portion of the sheath assembly 902. For example, the locking collar 920 can engage with the hub 906, including the proximal end 908 of the hub 906, such that the rotational orientation and translational position of the locking collar 920 is fixed relative to the sheath assembly 902. The rotational and translational lock of the locking collar 920 can engage with the medical device delivery assembly to reduce or prevent rotational and translational movement of the medical device delivery assembly relative to the locking collar 920, thereby reducing or preventing rotational and translational movement of the medical device delivery assembly relative to the sheath assembly 902. For example, the locking collar 920 can comprise a delivery lumen 922 extending therethrough to receive a corresponding portion of the medical device delivery assembly. FIG. 9 shows respective portions of the medical device delivery assembly disposed through the delivery lumen 922 of the locking collar 920 and the delivery lumen 904 of the sheath assembly 902. The rotational and translational lock can engage with a portion of the medical device delivery assembly extending through the delivery lumen 922 of the locking collar 920.

Referring to FIG. 9, the delivery lumen 922 of the locking collar 920 can be aligned, such as being coaxial, with the delivery lumen 904 of the sheath assembly 902 while the locking collar 920 is engaged with the sheath assembly 902. The hub 906 can comprise a delivery lumen 912, for example extending from the proximal end 908 to the distal end 910 of the hub 906, which forms a part of the delivery lumen 904 of the sheath assembly 902. In some instances, the hub 906 can comprise a seal 940 configured to form a hemostatic seal around a portion of the medical device delivery assembly extending through the delivery lumen 912 of the hub 906. The seal 940 can include a longitudinal lumen 942 extending therethrough, such as through a longitudinal dimension of the seal 940 parallel and/or coaxial with the longitudinal axis of the hub 906. The longitudinal lumen 942 can form a part of the delivery lumen 912 of the hub 906. The shaft 914 can comprise a delivery lumen 916 which forms another part of the delivery lumen 904 of the sheath assembly 902. A portion of the medical device delivery assembly can be positioned through the delivery lumen 922 of the locking collar 920, through the delivery lumen 912 of the hub 906, and then through the delivery lumen 916 of the shaft 914. In a locked state, the rotational and translational lock of the locking collar 920 can engage with a portion of medical device delivery assembly extending through its delivery lumen 922 to prevent or reduce rotation of and a proximal and distal translation of the medical device delivery assembly relative to the locking collar 920. The rotational and translational lock of the locking collar 920 can be configured to maintain a rotational orientation and translational position of the medical device delivery assembly relative to the locking collar 920. The locking collar 920 can be engaged with the sheath assembly 902 such that the rotational orientation and translational position of the locking collar 920 is maintained relative to the sheath assembly 902. In an unlocked state, the rotational and translational lock can be unengaged so as to allow rotational and translational movement of the medical device delivery assembly relative to the sheath assembly 902.

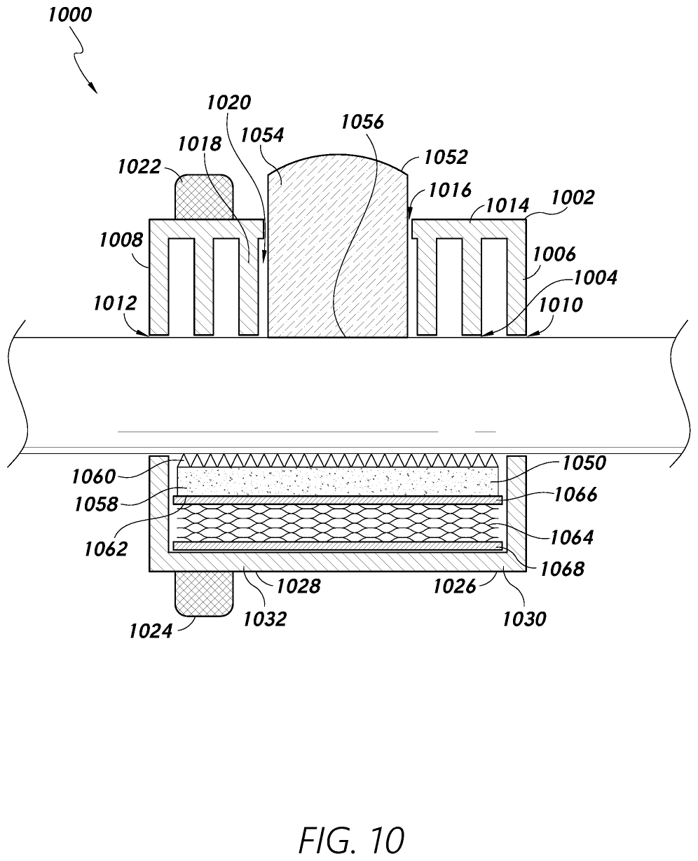

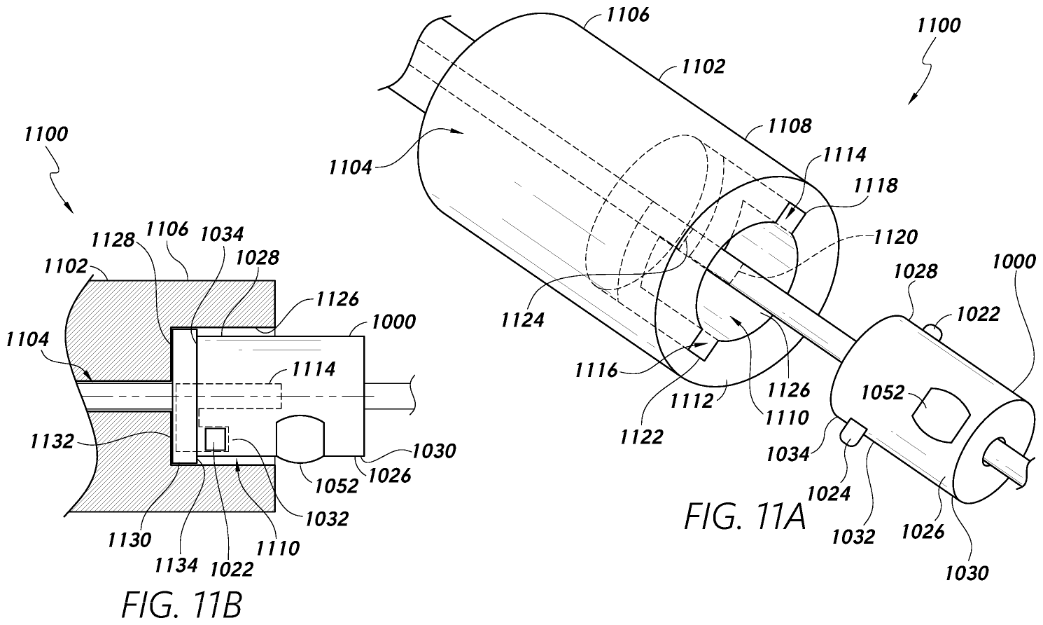

FIG. 10 shows an example of a locking collar 1000 and FIGS. 11A and 11B show an example of a medical delivery system 1100 comprising the locking collar 1000 of FIG. 10 and a sheath assembly 1102. The locking collar 1000 can engage with the sheath assembly 1102 such that a rotational orientation and translational position of the locking collar 1000 can be secured relative to that of the sheath assembly 1102. The locking collar 1000 can comprise a rotational and translational lock 1050 configured to engage with the medical device delivery assembly so as to prevent or reduce its rotation and translation, relative to the locking collar 1000, thereby preventing or reducing its rotation and translation relative to sheath assembly 1102.

FIG. 10 is a longitudinal cross-sectional view of the locking collar 1000. The locking collar 1000 can comprise a collar housing 1002. At least a portion of the rotational and translational lock 1050 can be disposed within the collar housing 1002. A delivery lumen 1004 can extend through the collar housing 1002. A portion of a medical device delivery assembly is shown as being disposed through the delivery lumen 1004 of the locking collar 1000. A first opening 1010 on proximal end 1006 and a second opening 1012 on a distal end 1008 of the collar housing 1002 can be aligned with proximal and distal ends of the delivery lumen 1004, respectively. The rotational and translational lock 1050 can comprise an elongate lever 1052 in a perpendicular or substantially perpendicular orientation relative to the delivery lumen 1004. The elongate lever 1052 can comprise a first user engagement end portion 1054 and a second contact member engagement end portion 1056 (not shown). The first user engagement end portion 1054 can extend externally of the collar housing 1002 through an opening 1016 on a lateral collar housing portion 1014 of the collar housing 1002. The lateral collar housing portion 1014 can be parallel or substantially parallel to the longitudinal axis of the collar housing 1002. The second contact member engagement end portion 1056 can be oriented toward the delivery lumen 1004. The lock 1050 can comprise a spring-loaded contact member 1058 received within the collar housing 1002. The spring-loaded contact member 1058 can comprise a delivery assembly engagement portion 1060 configured to be at a second opposing position about the delivery lumen 1004 relative to that of the second contact member engagement end portion 1056 of the elongate lever 1052. The spring-loaded contact member 1058 can be coupled to a spring 1064 configured to push the spring-loaded contact member 1058 toward the delivery lumen 1004. For example, in a relaxed state, the spring 1064 can be configured to maintain contact between a portion of the medical device delivery assembly extending through the delivery lumen 1004 and the delivery assembly engagement portion 1060 of the spring-loaded contact member 1058. In some instances, the spring 1064 can be coupled to a portion of the housing 1002, such as lateral collar housing portion 1014, such that the spring 1064 can be configured to push against a portion of the medical device delivery assembly in its relaxed state. For example, a first end 1066 of the spring 1064 can be coupled to a second opposing portion 1062 of the spring-loaded contact member 1058 having an opposing orientation relative to that of the delivery assembly engagement portion 1060. A second end 1068 of the spring 1064 can be coupled to the lateral collar housing portion 1014. The spring 1064 can comprise one or more of any number of different types of springs. In some instances, the spring 1064 can comprise a wave spring.

The elongate lever 1052 can be configured to be in a depressed state or a released state. In the depressed state, the elongate lever 1052 can be positioned further into the collar housing 1002 than in the released state. For example, in the unlocked state, the elongate lever 1052 can be configured to be in the depressed state such that the second contact member engagement end portion 1056 of the elongate lever 1052 can be in contact with the spring-loaded contact member 1058 to push the spring-loaded contact member 1058 away from the medical device delivery assembly. The delivery assembly engagement portion 1060 of the spring-loaded contact member 1058 can be spaced away from the medical device delivery assembly. In the locked state, the elongate lever 1052 can be configured to be in the released position. The elongate lever 1052 can be configured to be spaced away from the spring-loaded contact member 1058 while the elongate lever 1052 is in the released state such that the delivery assembly engagement portion 1060 of the spring-loaded contact member 1058 is allowed to contact a portion of the medical device delivery assembly extending within the delivery lumen 1004. In some instances, delivery assembly engagement portion 1060 of the spring-loaded contact member 1058 can comprise a texture configured to provide improved contact with the medical device delivery assembly, including a plurality of ridges and/or bumps. An operator, such as a surgeon, can manipulate the first user engagement end portion 1056 of the elongate lever 1052 to position the elongate lever 1052 in the depressed position or the released position. In some instances, the elongate lever 1052 can be spring-loaded such that the surgeon can push down on the elongate lever 1052 to position the elongate lever 1052 in the depressed state. The operator can push down the elongate lever 1052 again to position the elongate lever 1052 in the released state.

In some instances, the collar housing 1002 can comprise a perpendicular collar housing portion 1018 extending between the lateral collar housing portion 1014 and a portion of the delivery lumen 1004 of the collar housing 1002. The perpendicular collar housing portion 1018 can define a perpendicular lumen 1020. The opening 1016 on the lateral collar housing portion 1014 can be aligned with a first end of the perpendicular lumen 1020. A second end of the perpendicular lumen 1020 can open to the delivery lumen 1004. For example, the elongate lever 1052 can be partially received within the perpendicular lumen 1020 such that the elongate lever 1052 is positioned further into the perpendicular lumen 1020 in the depressed state as compared to the released state. In some instances, while the elongate lever 1052 is in the depressed state the second contact member engagement end portion 1056 can be in the delivery lumen 1004 such that it is in contact with the spring-loaded contact member 1058. In some instances, while the elongate lever 1052 is in the released state, the second contact member engagement end portion 1056 can be in the perpendicular lumen 1020 such that it is spaced from and not in contact with the spring-loaded contact member 1058.

As described herein, the locking collar 1000 can be configured to engage with a hub of a sheath assembly, such as a hub 1106 of a sheath assembly 1102 described with reference to FIGS. 11A and 11B. In some instances, the locking collar 1000 can comprise a first tab 1022 and a second tab 1024 extending laterally from the collar housing 1002, such as from a distal portion 1028 of the collar housing 1002. The first tab 1022 and second tab 1024 can be configured to engage with tab mating features of the hub 1106 so as to reduce or prevent the rotational orientation and translational position of the locking collar 1000 relative to that of the sheath assembly 1102. In some instances, the first and second tabs 1022, 1024 can extend from opposing positions on the distal portion 1028 of the collar housing 1002.

FIG. 11A is a perspective view of the medical delivery system 1100 comprising the locking collar 1000 described with reference to FIG. 10 and the sheath assembly 1102. FIG. 11B is a longitudinal cross-sectional view of a proximal portion of the sheath assembly 1102 and a side view of the locking collar 1000 engaged with the sheath assembly 1102. The sheath assembly 1102 can comprise a delivery lumen 1104 extending therethrough. FIGS. 11A and 11B show respective portions of a medical device delivery assembly disposed through the delivery lumen 1004 (not shown) of the locking collar 1000 and the delivery lumen 1104 of the sheath assembly 1102. Referring to FIG. 11A, the hub 1106 can comprise a housing 1108 at least partially defining a proximally oriented collar engaging recess 1110 configured to receive at least a part of the locking collar 1000. In some instances, a proximal end 1112 of the housing 1108 can at least partially define the collar engaging recess 1110. In some instances, the collar engaging recess 1110 can be configured to receive a portion of the locking collar 1000 such that a remaining portion of the locking collar 1000 can extend proximally from the collar engaging recess 1110. In some instances, a distal portion 1032 of the locking collar 1000 can be received within the collar engaging recess 1110 and a proximal portion 1030 of the locking collar 1000 can extend proximally from the collar engaging recess 1110. For example, the distal portion 1028 of the collar housing 1002 can be received within the collar engaging recess 1110 and a proximal portion 1026 of the collar housing 1002 can extend proximally from the collar engaging recess 1110.

As described herein, the first tab 1022 and second tab 1024 can extend laterally from the distal portion 1028 of the collar housing 1002. The collar engaging recess 1110 can comprise a first groove 1114 and a second groove 1116 configured to receive the first tab 1022 and the second tab 1024, respectively. The first groove 1114 and the second groove 1116 can be at respective positions on a lateral surface portion 1126 of the housing 1108 defining the collar engaging recess 1110. As shown in FIG. 11A, the first groove 1114 and the second groove 1116 can have respective first ends 1118, 1122 which opens to the proximal end 1112 of the collar housing 1108 to allow insertion of the first tab 1022 and the second tab 1024 into the respective grooves 1114, 1116. In some instances, the first groove 1114 and the second groove 1116 can be at opposing positions on the lateral surface portion 1126 and configured to receive the first tab 1022 and the second tab 1024 extending from opposing positions on the distal portion 1028 of the collar housing 1002. In some instances, the first and second grooves 1114, 1116 can each provide a non-linear path, for example comprising one or more bends. In some instances, each of the first groove 1114 and the second groove 1116 comprises a U-shaped path. In some instances, the first and second grooves 1114, 1116 can each comprise one or more curvatures. As described in further detail herein, the first and second tabs 1022, 1024 can be maintained against respective second ends 1120, 1124 of the first and second grooves 1114, 1116. The second ends 1120, 1124 do not open to the proximal end 1112 of the collar housing 1002. For example, the second ends 1120, 1124 can be on the lateral surface portion 1126 such that the first and second tabs 1022, 1024 can be maintained against respective second ends 1120, 1124. The one or more bends can facilitate maintaining the first and second tabs 1022, 1024 against respective second ends 1120, 1124 of the first and second grooves 1114, 1116. For example, at least a portion of the locking collar 1000 can be positioned within the collar engaging recess 1110 such that the first tab 1022 is inserted into the first groove 1114 through the first end 1118 of the first groove 1114 and the second tab 1024 is inserted into the second groove 1116 through the first end 1122 of the second groove 1116. The first and second tabs 1022, 1024 can be moved along the first and second grooves 1114, 1116, respectively, from the respective first ends 1118, 1122 to the respective second ends 1120, 1124 such that the first and second tabs 1022, 1024 can be maintained against the respective second ends 1120, 1124.

Referring to FIG. 11B, the hub 1106 can comprise a spring-loaded collar contacting member 1130 comprising at least a portion within the collar engaging recess 1110 such that the spring-loaded collar contacting member 1130 can be configured to be in contact with a distal end 1034 of the collar housing 1002 while the locking collar 1000 is engaged with the hub 1106. In some instances, the spring-loaded collar contacting member 1130 can be at a distal end 1128 of the collar engaging recess 1110. For example, a first surface 1132 of the spring-loaded collar contacting member 1130 can be oriented toward the distal end 1128 of the collar engaging recess 1110 and a second opposing surface 1134 can be oriented toward and be configured to contact the distal end 1034 of the collar housing 1002. In some instances, the spring-loaded collar contacting member 1130 be coupled to spring (not shown) such that the spring can be configured to push the spring-loaded collar contacting member 1130 against the distal end 1034 of the collar housing 1002.

In some instances, the spring-loaded collar contacting member 1130 pushing against the distal end 1034 of the collar housing 1002 can facilitate maintaining the first and second tabs 1022, 1024 against respective second ends 1120, 1124 of the first and second grooves 1114, 1116. For example, the first and second tabs 1022, 1024 of the locking collar 1000 can be aligned with respective first ends 1118, 1122 of the first and second grooves 1114, 1116 to allow positioning of the locking collar 1000 into the recess 1110. The first and second tabs 1022, 1024 can be advanced distally into the respective grooves 1114, 1116 as the locking collar 1000 is advanced distally into the collar engaging recess 1110. The first and second tabs 1022, 1024 can be advanced distally into the respective grooves 1114, 1116 and the locking collar 1000 can be advanced distally until the distal end 1034 of the collar housing 1002 presses against the spring-loaded collar contacting member 1130. For example, the locking collar 1000 can compress the spring coupled to the spring-loaded collar contacting member 1130. In some instances, the locking collar 1000 can be rotated about its longitudinal axis while the locking collar 1000 is at least partially received within the collar engaging recess 1110. In some instances, the locking collar 1000 can be rotated about its longitudinal axis while the spring-loaded collar contacting member 1130 is pressing against the distal end 1034 of the collar housing 1002 and the spring is compressed. The spring can then be allowed to push the locking collar 1000 proximally relative to the hub 1106 such that the first and second tabs 1022, 1024 can be moved along the first and second grooves 1114, 1116 to the respective second ends 1120, 1124 of the grooves 1114, 1116. In some instances, the spring-loaded collar contacting member 1130 can contact and push against the distal end 1034 of the collar housing 1002 such that the first and second tabs 1022, 1024 can be maintain against the respective second ends 1120, 1124 of the grooves 1114, 1116. In some instances, while the locking collar 1000 is engaged with the hub 1106, the first and second tabs 1022, 1024 can be maintained against the respective second ends 1120, 1124 to fix the rotational orientation and translational position of the locking collar 1000 relative to the sheath assembly 1102. In some instances, the proximal portion 1030 of the locking collar 1000 can extend proximally of the hub 1106 and the distal portion 1032 can be received within the collar engaging recess 1110, while the locking collar 1000 is engaged with the hub 1106. In some instances, the elongate lever 1052 can extend laterally from the collar housing 1002 proximally of the hub 1106 to facilitate manipulation of the elongate lever 1052 by an operator, while the locking collar 1000 is engaged with the hub 1106.

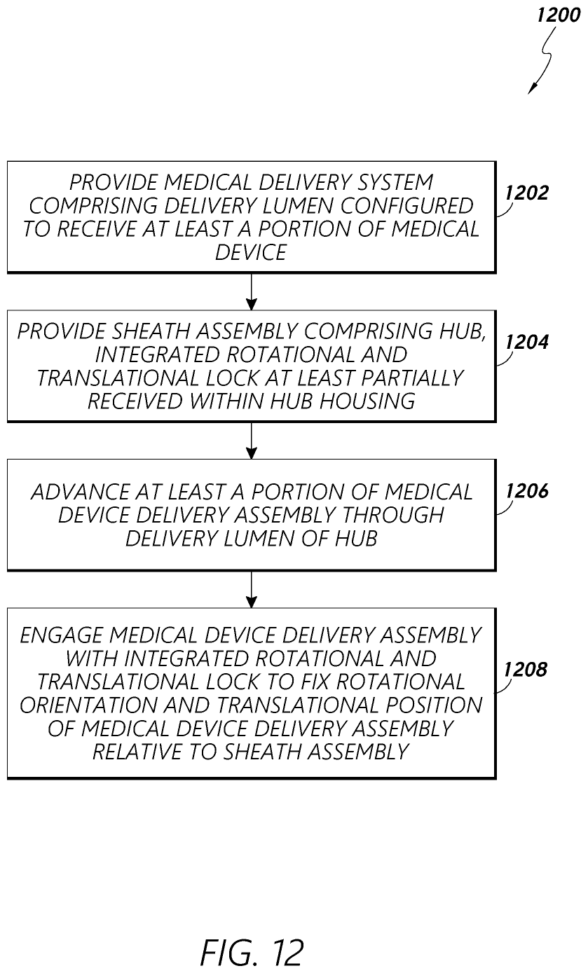

FIG. 12 is a process flow diagram of an example of a deployment process 1200 for delivering a medical device to a target site using a sheath assembly comprising one or more features as described herein. In block 1202, the process can involve providing a medical device delivery assembly comprising a delivery lumen extending therethrough. The delivery lumen can be configured to receive at least a portion of a medical device. In block 1204, the process can involve providing a sheath assembly. The sheath assembly can comprise one or more features of the sheath assembly 200, 300, 400, 500, 600, 700 described with reference to FIGS. 2 through 7. For example, the sheath assembly can comprise a hub that comprises a housing which has a delivery lumen extending therethrough and configured to receive a portion of the medical device delivery assembly. An integrated rotational and translational lock can be at least partially disposed within the housing. A shaft can extend distally from a distal end of the housing and comprise a delivery lumen extending therethrough. The delivery lumen of the shaft can be configured to receive another portion of the medical device delivery assembly.

In block 1206, the process can involve advancing at least a portion of the medical device delivery assembly through the delivery lumen of the hub. In some instances, a minimally invasive technique can be used. For example, the sheath assembly can be positioned into the patient using a trans-jugular approach, including at least a portion of the shaft of the sheath assembly. A corresponding portion of the medical device delivery assembly can then be advanced through the delivery lumen sheath assembly, including through the delivery lumen of the hub and the delivery lumen of the shaft.

In block 1208, the process can involve engaging the medical device delivery assembly with the integrated rotational and translational lock to fix a rotational orientation and translational position of the medical device delivery assembly relative to the sheath assembly. A corresponding portion of the medical delivery assembly can be advanced through the delivery lumen of the sheath assembly to position the medical device at a desired location within the patient. The integrated rotational and translational lock can then be activated to engage with a portion of the medical device delivery assembly to prevent rotational and translational movement of the medical device delivery assembly. In some instances, frictional contact between the integrated rotational and translational lock and the medical device delivery assembly can be configured to prevent rotational and translational movement of the medical device delivery assembly.

In some instances, engaging the medical device delivery assembly with the integrated rotational and translational lock comprises depressing an elongate lever extending from a lateral portion of the housing of the hub to contact the medical device delivery assembly with a delivery assembly engagement portion of the elongate lever. For example, the elongate lever can push the medical device delivery assembly against an engagement end of a spring-loaded contact member to position the medical device delivery assembly between the elongate lever and the spring-loaded contact member. The engagement end of the spring-loaded contact member can be at an opposing position about the delivery lumen relative to that of the engagement portion of the elongate lever. The medical device delivery assembly can be sandwiched between the delivery assembly engagement portion of the elongate lever and the engagement end of a spring-loaded contact member.

In some instances, engaging the medical device delivery assembly with the integrated rotational and translational lock can comprise rotating a proximal lock portion relative to the housing of the hub about a longitudinal axis of the housing. The lock can comprise a distal lock portion fixedly coupled to the housing. A tubular member comprising a flexible and noncompliant material can couple the proximal lock portion to the distal lock portion. A portion of the medical device delivery assembly can be configured to extend through an opening on the proximal lock portion, a longitudinal lumen of the tubular member and an opening on the distal lock portion. Turning the proximal lock portion relative to the housing can cause the flexible and noncompliant material of the tubular member to form a twisted portion. An inner surface defining the longitudinal lumen of the tubular member can contact the medical device delivery assembly extending through the twisted portion. Contact between the twisted portion of the tubular member and the medical device delivery assembly can provide sufficient frictional force to prevent rotational and translational movement of the medical device delivery assembly.

In some instances, engaging the medical device delivery assembly with the integrated rotational and translational lock comprises rotating a threaded rod member in a first direction about a longitudinal axis of the threaded rod member to advance the threaded rod member within a perpendicular lumen of the housing of the hub. The perpendicular lumen can be perpendicular or substantially perpendicular to a delivery lumen of the housing. The threaded rod member can be advanced into the perpendicular lumen until contact is made between a distal end of the threaded rod member and a portion of the medical device delivery assembly extending through the delivery lumen of the housing. The threaded rod member can push the medical device delivery assembly against one or more portions of the housing, such as one or more portions of the housing defining the delivery lumen such that sufficient frictional force is provided to prevent rotational and translational movement of the medical device delivery assembly.

In some instances, engaging the medical device delivery assembly with the integrated rotational and translational lock can comprise rotating a proximal lock portion in a first direction about a longitudinal axis of the housing to contact a distal end of a distally extending member of the lock with a tapered proximal end of a proximally extending member of the lock and to push the distal end of the distally extending member inward toward the longitudinal axis, causing the distally extending member to contact the medical device delivery assembly. The proximally extending member can be coupled to the housing of the sheath assembly hub. Rotating the proximal lock portion in the first direction can comprise rotating the proximal lock portion relative to the housing of the hub to advance the distally extending member toward the proximally extending member. For example, engaging the medical device delivery assembly with the integrated rotational and translational lock can comprise rotating the proximal lock portion to further screw the proximal lock portion onto the housing.

In some instances, engaging the medical device delivery assembly with the integrated rotational and translational lock can comprise mating a fastening feature of a strap having a first end associated with the housing with a mating fastening feature on the housing. The first end of the strap can be on a first side of an opening on the housing. The mating fastening feature can be on a second opposing side of the opening. The portion of the strap extending over the opening can contact an exposed portion of the medical device delivery assembly extending through the housing. Contact between the strap and the medical device delivery assembly can push the medical device delivery assembly against one or more portions of the housing. Sufficient frictional force can be provided by the contact between the medical device delivery assembly and the strap and the between the medical device delivery assembly and one or more portions of the housing to reduce or prevent rotational and translational movement of the medical device delivery assembly.

In some instances, engaging the medical device delivery assembly with the integrated rotational and translational lock can comprise inflating an inflatable member of the lock. The inflatable member can be inflated to achieve an inflated state. In the inflated state, an inner lumen surface defining a longitudinal lumen of the inflatable member can contact a corresponding portion of the medical device delivery assembly extending through the longitudinal lumen. Sufficient frictional force can be provided by the contact between the medical device delivery assembly and the inner lumen surface to reduce or prevent rotational and translational movement of the medical device delivery assembly.

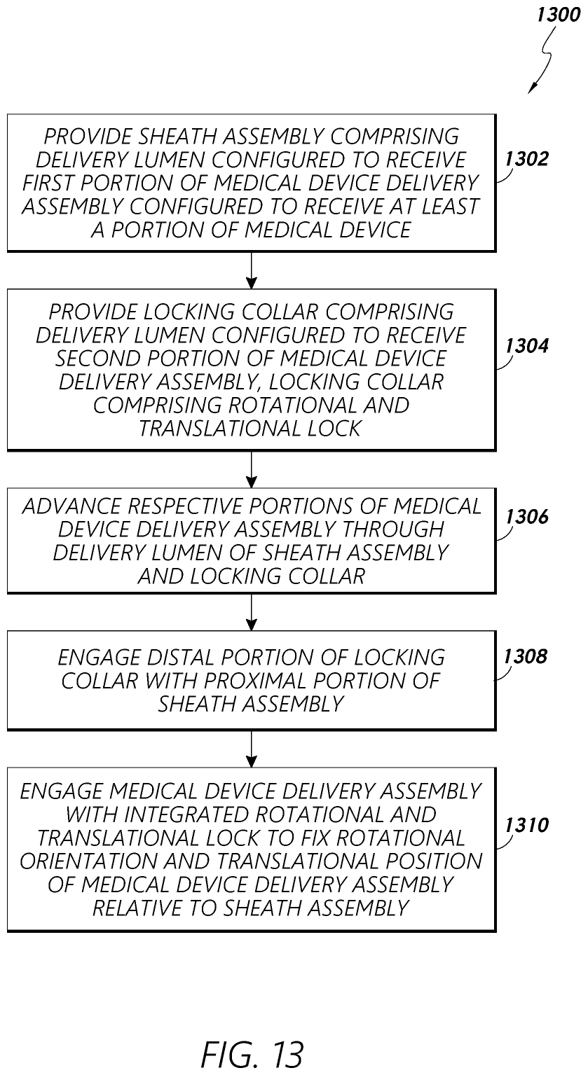

FIG. 13 is a process flow diagram of an example of a deployment process 1300 for delivering a medical device to a target site using a locking collar comprising one or more features as described herein. In block 1302, the process can involve providing a sheath assembly comprising a delivery lumen configured to receive a first portion of a medical device delivery assembly. The medical device delivery assembly can be configured to receive at least a portion of a medical device. In block 1304, the process can involve providing a locking collar comprising a delivery lumen configured to receive a second portion of the medical device delivery assembly, the locking collar comprising a rotational and translational lock. In a locked state, the rotational and translational lock can engage with the medical device delivery assembly to reduce or prevent rotational and translational movement of the medical device delivery assembly. In an unlocked state, the rotational and translational lock does not engage with the medical device delivery assembly such that the medical device delivery assembly can freely rotate around its longitudinal axis and translate proximally and distally relative to the sheath assembly.

In block 1306, the process can involve advancing respective portions of the medical device delivery assembly through the delivery lumens of the sheath assembly and locking collar. In some instances, respective portions of the medical device delivery assembly can be advanced through the delivery lumens of the sheath assembly and locking collar to position the medical device at a desired location within the patient. In some instances, the medical device can be configured to be delivered to a target site within the heart. As described herein, a minimally invasive technique can be used. For example, the sheath assembly can be positioned into the patient using a trans-jugular approach. A corresponding portion of the medical device delivery assembly can then be advanced through the sheath assembly that is at least partially positioned within the patient.

In block 1308, the process can involve engaging a distal portion of the locking collar with a proximal portion of the sheath assembly. Engaging the distal portion of the locking collar with a proximal portion of the sheath assembly can be configured to reduce or prevent translational and rotational movement of the locking collar relative to the sheath assembly. In some instances, the locking collar can be configured to engage with a hub of the sheath assembly. For example, engaging the distal portion of the locking collar with a proximal portion of the sheath assembly can comprise engaging the distal portion of the locking collar with a proximal portion of the hub, including a collar engaging recess on a distal end of the hub.

In some instances, engaging the distal portion of the locking collar with the proximal portion of the sheath assembly can comprises mating a first tab and a second tab of the locking collar with tab mating features of the hub. The first and second tabs can extend laterally from a locking collar housing of the locking collar. The first and second tabs can be at opposing positions on a distal portion of the locking collar housing. The tab mating features of the hub can comprise respective grooves for the first and second tabs.

In block 1310, the process can involve engaging the medical device delivery assembly with the rotational and translational lock to fix a rotational orientation and translational position of the medical device delivery assembly relative to the sheath assembly. In some instances, providing frictional contact between the medical device delivery assembly and the rotational and translational lock can prevent rotational and translational movement of the medical device delivery assembly.

In some instances, engaging the medical device delivery assembly with the rotational and translational lock can comprise releasing an elongate lever extending from a lateral portion of a locking collar housing to allow contact between a delivery assembly engagement portion of a spring-loaded contact member of the lock and a portion of the medical device delivery assembly extending within the delivery lumen of the locking collar.

### ADDITIONAL EXAMPLES

Example 1: A sheath assembly comprising:
a hub including:
   a housing having a delivery lumen extending therethrough, the delivery lumen being configured to receive a portion of a medical device delivery assembly;
   a seal disposed within the housing, the seal including a longitudinal lumen forming a part of the delivery lumen of the housing, the seal being configured to form a hemostatic seal around a corresponding portion of the medical device delivery assembly extending through the longitudinal lumen; and
   an integrated rotational and translational lock comprising at least a portion disposed within the housing proximally of the seal, the rotational and translational lock being configured to, in a locked state, engage with a corresponding portion of the medical device delivery assembly extending through the delivery lumen to prevent rotational and translational movement of the medical device delivery assembly relative to the sheath assembly and, in an unlocked state, to allow rotational and translational movement of the medical device delivery assembly relative to the sheath assembly.

Example 2: The assembly of any example herein, in particular example 1, wherein the medical device delivery assembly comprises a medical device delivery catheter.

Example 3: The assembly of any example herein, in particular example 1, wherein the medical device delivery assembly comprises a medical device loader configured to receive a medical device delivery catheter.

Example 4: The assembly of any example herein, in particular any one of examples 1 to 3, wherein:
the housing comprises a perpendicular housing portion proximal of the seal and extending between a lateral housing portion and the delivery lumen of the housing, the perpendicular housing portion defining a perpendicular lumen, an opening on the lateral housing portion being aligned with a first end of the perpendicular lumen and a second end of the perpendicular lumen opening to the delivery lumen of the housing; and
the rotational and translational lock comprises:
   an elongate lever partially received within the perpendicular lumen and configured to be in a depressed position or a released position, the elongate lever comprising a first user engagement end portion extending externally through the opening on the lateral portion of the housing and a second delivery assembly engagement end portion oriented toward the delivery lumen of the housing; and
   a spring-loaded contact member comprising an engagement end at an opposing position about the delivery lumen relative to that of the second end of the perpendicular lumen,
   wherein, in the locked state, the elongate lever is configured to be in the depressed position to cause the second delivery assembly engagement portion to push the medical device delivery assembly against the engagement end of the spring-loaded contact member, and in the unlocked state, the elongate lever is configured to be in the released position and the second delivery assembly engagement portion is not in contact with the medical device delivery assembly.

Example 5: The assembly of any example herein, in particular any one of examples 1 to 3, wherein the rotational and translational lock comprises:
a perpendicular lock portion proximal of the seal and extending between a lateral housing portion and the delivery lumen of the housing, the perpendicular lock portion defining a perpendicular lumen, an opening on the lateral housing portion being aligned with a first end of the perpendicular lock portion and a second end of the perpendicular lock portion opening to the delivery lumen of the housing and an inner lumen surface defining the perpendicular lumen having threads along at least a portion thereof; and
a threaded rod member partially received within the perpendicular lumen and comprising threads on a lateral surface portion, the threads being configured to mate with the threads on the inner lumen surface defining the perpendicular lumen;
wherein the threaded rod member is configured to be rotated in a first direction about a longitudinal axis of the rod to advance the threaded rod member within the perpendicular lumen and cause contact between a distal end of the threaded rod member and the medical device delivery assembly to achieve the locked state, and
wherein the threaded rod member is configured to be rotated in a second opposing direction about the longitudinal axis of the rod to withdraw the rod away from the medical device delivery assembly and achieve the unlocked state.

Example 6: The assembly of any example herein, in particular example 5, wherein the threads of the threaded rod member and the inner lumen surface each comprise a pitch configured to allow a quarter turn in the first direction and the second opposing direction to alternate between the locked state and the unlocked state.

Example 7: The assembly of any example herein, in particular any one of examples 1 to 3, wherein the rotational and translational lock comprises:
a proximal lock portion comprising a lateral surface oriented toward a longitudinal axis of the housing and an externally oriented lateral surface of the housing, the lateral surface of the proximal lock portion comprising threads configured to mate with corresponding threads on the externally oriented lateral surface of the housing;
a distal lock portion fixedly coupled to the housing; and
a tubular member comprising a noncompliant material coupling the proximal and distal lock portions, a longitudinal lumen of the tubular member forming a portion of the delivery lumen of the housing,
wherein rotating the proximal lock portion relative to the housing about the longitudinal axis of the housing is configured to cause the noncompliant material of the tubular member to form a twisted portion, an inner surface defining the longitudinal lumen of the tubular member being configured to contact the medical device delivery assembly extending through the twisted portion to thereby achieve the locked state.

Example 8: The assembly of any example herein, in particular any one of examples 1 to 3, wherein the rotational and translational lock comprises:
a proximal lock portion defining a distally oriented recess configured to receive a distal portion of the housing, the proximal lock portion further comprising:
   a lateral surface defining the distally oriented recess and oriented toward a longitudinal axis of the housing, the lateral surface of the proximal lock portion comprising threads configured to mate with corresponding threads on an externally oriented lateral surface of the housing, and
   a distally extending member extending from a distally oriented surface of the recess, the distally extending member defining a first longitudinal lumen forming a portion of the delivery lumen of the housing; and
a distal lock portion fixedly coupled to the housing, the distal lock portion comprising a proximally protruding member comprising a second longitudinal lumen forming another portion of the delivery lumen and a proximal end of the proximally extending member tapering inward toward a longitudinal axis of the housing,
wherein rotation of the proximal lock portion in a first direction about the longitudinal axis of the housing causes contact of a distal end of the distally extending member with the tapered proximal end of the proximally extending member to thereby push the distal end of the distally extending member inward toward the longitudinal axis and contact the medical device delivery assembly and achieve the locked state.

Example 9: The assembly of any example herein, in particular example 8, wherein at least a portion of an inner lumen surface defining the first longitudinal lumen comprises a texture.

Example 10: The assembly of any example herein, in particular example 9, wherein the texture comprises ribs.

Example 11: The assembly of any example herein, in particular example 9 or 10, wherein the texture comprises glass fibers.

Example 12: The assembly of any example herein, in particular any one of examples 1 to 3, wherein:
a lateral housing portion comprises an opening, the opening being in communication with the delivery lumen of the housing and the opening being configured to expose a portion of the medical device delivery assembly extending through the delivery lumen; and
the rotational and translational lock comprises:
   a strap comprising a first end associated with the housing on a first side of the opening and a second free end, the strap comprising a plurality of fastening features regularly spaced along at least a portion of a length of the strap; and
   a mating fastening feature on the housing on a second opposing side of the opening,
   wherein, in the locked state, the mating fastening feature on the housing is configured to be coupled to a fastening feature of the strap and a portion of the strap extending over the opening is configured to contact the exposed portion of medical device delivery assembly to prevent rotational and translational movement of the medical device delivery assembly.

Example 13: The assembly of any example herein, in particular example 12, wherein the mating fastening feature comprises a protrusion and the plurality of fastening features comprises a plurality of openings, at least a portion of the protrusion being configured to extend through an opening of the strap in the locked state.

Example 14: The assembly of any example herein, in particular any one of examples 1 to 3, wherein the rotational and translational lock comprises an inflatable member received within the housing and defining a lumen extending therethrough, the inflatable member comprising:
an injection port aligned with an opening on a lateral portion of the housing, the injection portion being configured to provide fluid communication with an inner volume of the inflatable member,
wherein, the lumen of the inflatable member forms a portion of the delivery lumen of the housing, and
wherein, in the locked state, the inflatable member is configured to be in an inflated state to cause an inner surface defining the lumen of the inflatable member to contact a corresponding portion of the medical device delivery assembly extending therethrough.

Example 15: The assembly of any example herein, in particular example 14, further comprising an injection device configured to mate with the injection port of the inflatable member to provide and withdraw fluid from the inflatable member.

Example 16: The assembly of any example herein, in particular any one of examples 1 to 15, wherein the sheath assembly is sterilized.

Example 17: A medical delivery system comprising:
a sheath assembly comprising a delivery lumen configured to receive a first portion of a medical device delivery assembly; and
a locking collar configured to engage with a proximal portion of the sheath assembly and comprising a delivery lumen configured to receive a second portion of a medical device delivery assembly,
wherein the locking collar comprises a rotational and translational lock configured to, in a locked state, engage with a portion of the medical device delivery assembly extending through the delivery lumen of the locking collar to prevent a rotational and a translational movement of the medical device delivery assembly relative to the sheath assembly and, in an unlocked state, to allow rotational and translational movement of the medical device delivery assembly relative to the sheath assembly.

Example 18: The system of any example herein, in particular example 17, wherein the locking collar comprises:
a collar housing comprising a perpendicular collar housing portion extending between a lateral collar housing portion and a portion of the delivery lumen of the collar housing, the perpendicular housing portion defining a perpendicular lumen, an opening on the lateral collar housing portion being aligned with a first end of the perpendicular lumen and a second end of the perpendicular lumen opening to the delivery lumen of the collar housing;
an elongate lever partially received within the perpendicular lumen and configured to be in a depressed position or a released position, the elongate lever comprising a first user engagement end portion extending externally through the opening on the lateral portion of the collar housing and a second contact member engagement end portion oriented toward the delivery lumen; and
a spring-loaded contact member received within the collar housing and comprising a medical device engagement portion configured to be in contact with the medical device extending through the delivery lumen, the medical device engagement portion being at a second opposing position about the delivery lumen relative to that of the second end of the perpendicular lumen,
wherein, in the locked state, the lever is configured to be in the released position to allow contact between the medical device engagement portion of the spring-loaded contact member and a portion of the medical device delivery assembly extending within the delivery lumen, and
wherein, in the unlocked state, the lever is configured to be in the depressed state, the second contact member engagement portion being in contact with the spring-loaded contact member to push the spring-loaded contact member away from the medical device.

Example 19: The system of any example herein, in particular example 18, wherein the spring-loaded contact member is coupled to a wave spring.

Example 20: The system of any example herein, in particular any one of examples 17 to 19, wherein the locking collar comprises a first tab and a second tab extending laterally from a distal portion of the collar housing at opposing positions, the first and second tabs being configured to engage with tab mating features of the hub.

Example 21: The system of any example herein, in particular example 20, wherein the housing of the hub comprises:
a proximal housing portion defining a proximally oriented collar engaging recess on a proximal end of the housing, the recess being configured to receive a distal portion of the locking collar;
a first groove and a second groove at opposing positions on a lateral surface portion defining the recess, the first groove being configured to receive the first tab and the second groove being configured to receive the second tab; and
a spring-loaded collar contacting member at a distal end of the collar engaging recess and configured to be in contact with a distal end of the locking collar,
wherein the spring-loaded collar contacting member is configured to push against the distal end of the locking collar received in the collar engaging recess to maintain the first tab and the second tab in the first and second grooves, respectively.

Example 22: The system of any example herein, in particular example 21, wherein the first groove defines a first path comprising a curvature and the second groove defines a second path comprising another curvature.

Example 23: The system of any example herein, in particular example 22, wherein each of the first groove and the second groove defines a U-shaped path.

Example 24: The system of any example herein, in particular any one of examples 17 to 23, wherein the medical device delivery assembly comprises a medical device delivery catheter.

Example 25: The system of any example herein, in particular any one of examples 17 to 24, wherein the medical device delivery assembly comprises a medical device loader configured to receive a medical device delivery catheter.

Example 26: The system of any example herein, in particular any one of examples 17 to 25, wherein at least one of the sheath assembly and the locking collar are sterilized.

Example 27: A medical delivery system comprising:
a medical device loader comprising:
   a first delivery lumen configured to receive a medical device delivery catheter, and
   a first integrated rotational and translational lock configured to, while in a locked state, engage with a portion of the medical device delivery catheter extending through the first delivery lumen of the medical device loader to prevent rotational and translational movement of the medical device delivery catheter relative to the medical device loader, and, while in an unlocked state, allow rotational and translational movement of the medical device delivery catheter relative to the medical device loader;
a sheath assembly comprising:
   a second delivery lumen configured to receive a portion of the medical device loader, and
   a second integrated rotational and translational lock configured to, while in a locked state, engage with a portion of the medical device loader extending through the second delivery lumen of the sheath assembly to prevent rotational and translational movement of the medical device loader relative to the sheath assembly, and, while in an unlocked state, allow rotational and translational movement of the medical device loader relative to the sheath assembly.

Example 28: The system of any example herein, in particular example 27, wherein the medical device loader comprises:
a housing comprising a perpendicular housing portion extending between a lateral housing portion and a delivery lumen of the housing forming at least a portion of the first delivery lumen, the perpendicular housing portion defining a perpendicular lumen, an opening on the lateral housing portion being aligned with a first end of the perpendicular lumen and a second end of the perpendicular lumen opening to the delivery lumen of the housing; and
the first rotational and translational lock comprises:
   an elongate lever partially received within the perpendicular lumen and configured to be in a depressed position or a released position, the elongate lever comprising a first user engagement end portion extending externally through the opening on the lateral portion of the housing and a second delivery catheter engagement end oriented toward the delivery lumen of the housing; and
   spring-loaded contact member comprising an engagement end at an opposing position about the delivery lumen relative to that of the second end of the perpendicular lumen,
   wherein, in the locked state, the elongate lever is configured to be in the depressed position to cause the second delivery catheter engagement end to push the medical device delivery catheter against the engagement end of the spring-loaded contact member, and in the unlocked state, the elongate lever is configured to be in the released position and the second delivery catheter engagement end is not in contact with the medical device delivery catheter.

Example 29: The system of any example herein, in particular example 27, wherein the medical device loader comprises:
a housing comprising a perpendicular housing portion extending between a lateral housing portion and a delivery lumen of the housing forming at least a portion of the first delivery lumen, the perpendicular housing portion defining a perpendicular lumen, an opening on the lateral housing portion being aligned with a first end of the perpendicular lumen and a second end of the perpendicular lumen opening to the delivery lumen of the housing; and
the first rotational and translational lock comprises:
   an elongate lever partially received within the perpendicular lumen and configured to be in a depressed position or a released position, the elongate lever comprising a first user engagement end portion extending externally through the opening on the lateral portion of the housing and a second delivery catheter engagement end oriented toward the delivery lumen of the housing; and
   spring-loaded contact member comprising an engagement end at an opposing position about the delivery lumen relative to that of the second end of the perpendicular lumen,
   wherein, in the locked state, the elongate lever is configured to be in the released position to cause the second delivery catheter engagement end to be spaced from spring-loaded contact member, and in the unlocked state, the elongate lever is configured to be in the depressed position and the second delivery catheter engagement end is in contact with the spring-loaded contact member.

Example 30: The system of any example herein, in particular any one of examples 27 to 29, wherein at least one of the medical device loader and sheath assembly are sterilized.

Example 31: A method of delivering a medical device, the method comprising:
providing a medical device delivery assembly comprising a delivery lumen extending therethrough and configured to receive at least a portion of a medical device;
providing a sheath assembly, the sheath assembly comprising a hub with a housing, a delivery lumen extending through the housing and configured to receive a portion of the medical device delivery assembly, and an integrated rotational and translational lock at least partially received within the housing;
advancing at least a portion of the medical device delivery assembly through the delivery lumen of the housing; and
engaging the medical device delivery assembly with the integrated rotational and translational lock to fix a rotational orientation and translational position of the medical device delivery assembly relative to the sheath assembly.

Example 32: The method of any example herein, in particular example 31, wherein engaging the medical device delivery assembly with the integrated rotational and translational lock comprises depressing an elongate lever extending from a lateral portion of the housing of the hub to contact the medical device delivery assembly with a delivery system engagement portion of the elongate lever and push the medical device delivery assembly against an engagement end of a spring-loaded contact member at an opposing position about the delivery lumen relative to that of the engagement portion of the elongate lever.

Example 33: The method of any example herein, in particular example 31, wherein engaging the medical device delivery assembly with the integrated rotational and translational lock comprises rotating a proximal lock portion relative to the housing of the hub about a longitudinal axis of the housing to cause a noncompliant material of a tubular member of the integrated rotational and translational lock to form a twisted portion, an inner surface defining a longitudinal lumen of the tubular member contacting the medical device delivery assembly extending through the twisted portion.

Example 34: The method of any example herein, in particular example 31, wherein engaging the medical device delivery assembly with the integrated rotational and translational lock comprises rotating a threaded rod member in a first direction about a longitudinal axis of the threaded rod member to advance the threaded rod member within a perpendicular lumen of the housing of the hub and contact a distal end of the threaded rod member with the medical device delivery assembly, the perpendicular lumen being perpendicular to the delivery lumen of the housing.

Example 35: The method of any example herein, in particular example 31, wherein engaging the medical device delivery assembly with the integrated rotational and translational lock comprises rotating a proximal lock portion in a first direction about a longitudinal axis of the housing to contact a distal end of a distally extending member of the lock with a tapered proximal end of a proximally extending member of the lock and to push the distal end of the distally extending member inward toward the longitudinal axis, causing the distally extending member to contact the medical device delivery assembly.

Example 36: The method of any example herein, in particular example 31, wherein engaging the medical device delivery assembly with the integrated rotational and translational lock comprises inflating an inflatable member of the lock to achieve an inflated state and cause an inner surface defining a lumen of the inflatable member to contact a corresponding portion of the medical device delivery assembly extending through the lumen.

Example 37: The method of any example herein, in particular example 31, wherein engaging the medical device delivery assembly with the integrated rotational and translational lock comprises mating a fastening feature of a strap comprising a first end associated with the housing on a first side of an opening on the housing with a mating fastening feature on a second side of the opening.

Example 38: A method of delivering a medical device, the method comprising:
providing a sheath assembly comprising a delivery lumen configured to receive a first portion of a medical device delivery assembly configured to receive at least a portion of a medical device;
providing a locking collar comprising a delivery lumen configured to receive a second portion of the medical device delivery assembly, the locking collar comprising a rotational and translational lock;
advancing respective portions of the medical device delivery assembly through the delivery lumen of the sheath assembly and locking collar;
engaging a distal portion of the locking collar with a proximal portion of the sheath assembly; and
engaging the medical device delivery assembly with the rotational and translational lock to fix a rotational orientation and translational position of the medical device delivery assembly relative to the sheath assembly.

Example 39: The method of any example herein, in particular example 38, wherein engaging the medical device delivery assembly with the rotational and translational lock comprises releasing an elongate lever extending from a lateral portion of a locking collar housing to allow contact between a medical device engagement portion of a spring-loaded contact member of the lock and a portion of the medical device delivery assembly extending within the delivery lumen of the locking collar.

Example 40: The method of any example herein, in particular example 38, wherein engaging the distal portion of the locking collar with the proximal portion of the sheath assembly comprises mating a first tab and a second tab extending laterally and at opposing positions from a distal portion of a collar housing with tab mating features of a hub of the sheath assembly.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other embodiment(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A sheath assembly comprising:
a hub including:
a housing having a delivery lumen extending therethrough, the delivery lumen being configured to receive a portion of a medical device delivery assembly;
a seal disposed within the housing, the seal including a longitudinal lumen forming a part of the delivery lumen of the housing, the seal being configured to form a hemostatic seal around a corresponding portion of the medical device delivery assembly extending through the longitudinal lumen; and
an integrated rotational and translational lock comprising at least a portion disposed within the housing proximally of the seal, the rotational and translational lock being configured to, in a locked state, engage with a corresponding portion of the medical device delivery assembly extending through the delivery lumen to prevent rotational and translational movement of the medical device delivery assembly relative to the sheath assembly and, in an unlocked state, to allow rotational and translational movement of the medical device delivery assembly relative to the sheath assembly.

2. The assembly of claim 1, wherein the medical device delivery assembly comprises at least one of a medical device delivery catheter and a medical device loader configured to receive the medical device delivery catheter.

3. The assembly of claim 1, wherein the rotational and translational lock comprises:
a proximal lock portion defining a distally oriented recess configured to receive a distal portion of the housing, the proximal lock portion further comprising:
a lateral surface defining the distally oriented recess and oriented toward a longitudinal axis of the housing, the lateral surface of the proximal lock portion comprising threads configured to mate with corresponding threads on an externally oriented lateral surface of the housing, and
a distally extending member extending from a distally oriented surface of the recess, the distally extending member defining a first longitudinal lumen forming a portion of the delivery lumen of the housing; and
a distal lock portion fixedly coupled to the housing, the distal lock portion comprising a proximally extending member comprising a second longitudinal lumen forming another portion of the delivery lumen and a proximal end of the proximally extending member tapering inward toward a longitudinal axis of the housing,
wherein rotation of the proximal lock portion in a first direction about the longitudinal axis of the housing causes contact of a distal end of the distally extending member with the tapered proximal end of the proximally extending member to thereby push the distal end of the distally extending member inward toward the longitudinal axis and contact the medical device delivery assembly and achieve the locked state.

4. The assembly of claim 3, wherein at least a portion of an inner lumen surface defining the first longitudinal lumen comprises a texture.

5. The assembly of claim 4, wherein the texture comprises ribs.

6. The assembly of claim 4, wherein the texture comprises glass fibers.

7. The assembly of claim 3, wherein rotation of the proximal lock portion in the first direction about the longitudinal axis of the housing is configured to distally advance the proximal lock portion toward the housing and allow screwing of the proximal lock portion onto the housing using the threads of the proximal lock portion and the corresponding threads of the housing.

8. The assembly of claim 7, wherein screwing of the proximal lock portion onto the housing is configured to cause bending inward of a distal portion of the distally extending member and allowing the integrated rotational and translational lock to achieve the locked state.

9. The assembly of claim 3, wherein rotation of the proximal lock portion in a second direction about the longitudinal axis of the housing is configured to proximally displace the proximal lock portion away from the distal end of the housing and allow unscrewing of the proximal lock portion from the housing using the threads of the proximal lock portion and the corresponding threads of the housing.

10. The assembly of claim 9, wherein unscrewing of the proximal lock portion from the housing is configured to remove bending in a distal portion of the distally extending member, allowing the integrated rotational and translational lock to achieve the unlocked state.

11. The assembly of claim 3, wherein a diameter of the second longitudinal lumen is larger than that of the first longitudinal lumen to allow bending inward of a distal portion of the distally extending member.

12. The assembly of claim 3, wherein a distal portion of the distally extending member comprises a texture comprising at least one of ribs, dots and glass fibers.

13. A method of delivering a medical device, the method comprising:
providing a medical device delivery assembly comprising a delivery lumen extending therethrough and configured to receive at least a portion of a medical device;
providing a sheath assembly, the sheath assembly comprising a hub with a housing, a delivery lumen extending through the housing and configured to receive a portion of the medical device delivery assembly, and an integrated rotational and translational lock at least partially received within the housing;
advancing at least a portion of the medical device delivery assembly through the delivery lumen of the housing; and
engaging the medical device delivery assembly with the integrated rotational and translational lock to fix a rotational orientation and translational position of the medical device delivery assembly relative to the sheath assembly.

14. The method of claim 13, wherein engaging the medical device delivery assembly with the integrated rotational and translational lock comprises rotating a proximal lock portion in a first direction about a longitudinal axis of the housing to contact a distal end of a distally extending member of the lock with a tapered proximal end of a proximally extending member of the lock and to push the distal end of the distally extending member inward toward the longitudinal axis, causing the distally extending member to contact the medical device delivery assembly.

15. The method of claim 14, wherein causing the distally extending member to contact the medical device delivery assembly comprises bending a distal portion of the distally extending member inward toward the longitudinal axis.

16. The method of claim 14, wherein rotating the proximal lock portion in the first direction comprises rotating the proximal lock portion relative to the housing to advance the proximal lock portion toward the housing to screw the proximal lock portion onto the housing.

17. The method of claim 16, further comprising rotating the proximal lock portion in a second direction about the longitudinal axis of the housing to unscrew the proximal lock portion from the housing and remove bending in the distal portion of the distally extending member.

18. The method of claim 14, wherein causing the distally extending member to contact the medical device delivery assembly comprises causing a distal portion of the distally extending member to contact the medical device delivery assembly, the distal portion comprising a texture.

19. The method of claim 18, wherein causing the distal portion of the distally extending member to contact the medical device delivery assembly comprises causing a distal portion comprising at least one of ribs and dots to contact the medical device delivery assembly.

20. The method of claim 18, wherein causing the distal portion of the distally extending member to contact the medical device delivery assembly comprises causing a distal portion comprising glass fibers to contact the medical device delivery assembly.
